(19) United States
(12) Patent Application Publication
(10) Pub. No.: US 2011/0177574 A1
(43) Pub. Date: Jul. 21, 2011

(54) Method and Apparatus for Processing Waste Containing Fermentable Raw Material

(76) Inventors: Mikko Ahokas, Oulu (FI); Antti Pasanen, Espoo (FI); Reetta Niemi-Korpi, Vantaa (FI)

(21) Appl. No.: 12/993,638

(22) PCT Filed: May 11, 2009
(86) PCT No.: PCT/FI09/50381

§ 371 Date: Apr. 4, 2011

(30) Foreign Application Priority Data
May 20, 2008 (FI) 20085477

(51) Int. Cl. C12P 7/10; C12M 1/00

(52) U.S. Cl. 435/165; 435/290.1

(57) ABSTRACT

The invention relates to a method and an apparatus for processing waste (1) containing fermentable raw materials selected from sugars and raw materials such as starch and cellulose capable of being saccharified into fermentable sugars. The method includes a crushing step for forming fine-divided waste (30), a saccharification step for saccharifying with saccharifying enzymes (20) the fine-divided waste (30) to obtain saccharified fine-divided waste (31), a fermentation step for fermenting with an ethanol-producing microorganism (23) said saccharified fine-divided waste (31) to obtain fermented fine-divided waste (32) containing mixture of ethanol and water (25), and a vaporization step for at least partly separating by vaporization said mixture of ethanol and water (25), a dehydrating step for at least partly dehydrating said fermented fine-divided waste (32) to form dry matter (24).

### FIELD OF THE INVENTION

The invention relates to a method for processing waste containing fermentable raw materials selected from sugars and raw materials such as starch and cellulose capable of being saccharified into fermentable sugars as defined in the preamble of independent claim 1.

The invention also relates to an apparatus for processing waste containing fermentable raw materials selected from sugars and raw materials such as starch and cellulose capable of being saccharified into fermentable sugars as defined in the preamble of independent claim 40.

The invention also relates to the use of the method or the apparatus for the production of ethanol from municipal biodegradable waste.

The invention relates to processing waste containing fermentable raw material selected from sugars and raw materials such as starch and cellulose capable of being saccharified into fermentable sugars.

The invention relates especially, but not excluding other types of waste containing fermentable raw material selected from sugars and raw materials such as starch and cellulose capable of being saccharified into fermentable sugar, to processing of municipal biodegradable waste. By municipal biodegradable waste is meant municipal biodegradable waste containing biodegradable waste, which means that the (so-called) municipal biodegradable waste to be processed by the method and apparatus need not to be entirely biodegradable and that the municipal biodegradable waste to be processed by the method and the apparatus can contain liquid matter and that the municipal biodegradable waste to be processed by the method and apparatus can contain non-biodegradable objects.

Nowadays municipal waste is sorted by the producer into several categories, one of which is municipal biodegradable waste including for example food waste and other similar biodegradable organic matter. The municipal biodegradable waste is normally placed by the producer in special waste bins for biodegradable material. Thereafter the municipal biodegradable waste is collected from the special waste bins and transported to processing facilities for processing. Known biodegradable waste processing methods are composting, biodegradation and burning. Composting means shortly aerobic decomposition of biodegradable waste to produce compost. In biodegrading the municipal biodegradable waste is broken down by the enzymes produced by living organisms.

Municipal biodegradable waste can in addition be used as a raw material for the production of biofuels. According to a research conducted by VTT (Technical Research Centre of Finland) 25 to 30 w % of the biodegradable waste in Finland is dry matter, the remaining 70 to 75 w % is liquid. Total carbohydrate content is about 40% of the dry matter, about 20% of the dry matter is starch, and about 10% ash. The rest is proteins, lignins and lipids WO 99/06133 presents a process for the production of ethanol and solid biofuel from municipal waste and other cellulosic feedstock.

Publication WO 2007/036795 discloses a process for production of fermentation products, including bioethanol by non-pressurised pre-treatment, enzymatic hydrolysis and fermentation of waste fractions containing mono- and/or polysaccharides, having a relatively high dry matter content. The process in its entirety, i.e. from non-pressurised pre-treatment over enzymatic hydrolysis and fermentation to sorting of fermentable and non-fermentable solids can be processed at a relatively high dry matter content in a single vessel or similar device using free fall mixing for the mechanical processing of the waste fraction.

Publication U.S. Pat. No. 5,677,154 discloses a method and an apparatus for the production of ethanol from non-virgin biomass having deleterious materials therein is provided. The deleterious materials prevent or retard the production of ethanol when the non-virgin biomass is combined with a fermentation material. The method includes primary processing, secondary processing and/or blending the non-virgin biomass with virgin biomass such that the effect of the deleterious materials is reduced, thereby allowing production of ethanol when the biomass is combined with the fermentation material.

Publication JP 2007097422 (A) discloses a system for fermentation, distillation and drying that is equipped with one closed type fermentation and drying tank, a suction pump for sucking gas in the closed type fermentation and drying tank and depressurizing the tank and an evaporator which communicates with the closed type fermentation and drying tank and condenses gases produced in treatment processes of water adjustment, saccharification, solid fermentation, distillation and drying of an organic substance stored in the closed type fermentation and drying tank and converting the gases to a liquid.

### OBJECTIVE OF THE INVENTION

The object of the invention is to provide a new and inventive simple method and apparatus for processing waste.

### SHORT DESCRIPTION OF THE INVENTION

The method of the invention for processing waste is characterized by the definitions of independent claim 1.

Preferred embodiments of the method are defined in the dependent claims 2 to 39.

The apparatus of the invention for processing waste is correspondingly characterized by the definitions of independent claim 40.

Preferred embodiments of the apparatus are defined in the dependent claims 41 to 68.

The invention also relates to the use of the method or the apparatus for the production of ethanol from municipal biodegradable waste.

In the invention the waste containing fermentable raw material selected from sugars and raw materials such as starch and cellulose capable of being saccharified into fermentable sugars is at least partly crushed to produce fine-divided waste of the waste. Said fine-divided waste is thereafter at least partly saccharified with saccharifying enzymes to break down starch and/or cellulose of the fine-divided waste to produce saccharified fine-divided waste containing fermentable sugars. Said saccharified fine-divided waste containing fermentable sugars is thereafter at least partly fermented with ethanol-producing microbes such as yeast to produce fermented fine-divided waste containing a mixture of ethanol and water. Said fermented fine-divided waste containing fermentable sugars and mixture of ethanol and water is thereafter at least partly dehydrated in an vaporization and dehydrating means for at least partly separating by evaporation said mixture of ethanol and water from said fermented fine-divided waste and for dehydrating said fermented fine-divided waste to produce dry matter of the fermented fine-divided waste. Said evaporated mixture of ethanol and water and said dry matter are thereafter discharged from said vaporization and dehydrating means.

The invention may comprise feeding partly dehydrated fermented fine-divided waste (partly evaporated sludge formed) from said vaporization and dehydrating means back to the fermentation means For example in situations of fermentation inhibition in the fermentation vessel, caused by high ethanol concentration or high organic acid concentration (e.g. lactic acid and acetic acid) or other fermentation inhibitors, it is possible to feed fermented fine-divided waste from the fermentation vessel to the vaporization and dehydrating vessel to perform a pre-vaporization step in which for a certain period of time excess volatile inhibiting compounds, such as ethanol, lactic acid and acetic acid, is evaporated off fermented fine-divided waste with the vaporization and dehydrating vessel, after which partly evaporated fermented fine-divided waste that has a reduced amount of fermentation inhibitors is fed back to the fermentation vessel for further or additional fermentation of the partly evaporated fermented fine-divided waste, resulting in higher yield and fermentation rate.

The fermentation step of the method of the invention may for example comprise a pre-vaporization step for removing fermentation inhibitors from fermented fine-divided waste containing fermentation inhibitors by vaporization fermentation inhibitors from said fermented fine-divided waste containing fermentation inhibitors.

The fermentation step of the method of the invention may for example comprise a pre-vaporization step which includes feeding step for firstly feeding fermented fine-divided waste from a fermentation vessel to a vaporization and dehydration vessel to perform to a pre-evaporation step to evaporate fermentation inhibitors such as excess volatile inhibiting compounds, such as ethanol, lactic acid and acetic acid, which inhibits fermentation in the fermentation vessel off from fermented fine-divided waste with the vaporization and dehydrating vessel, and comprise a feeding step for after said pre-evaporation step feeding back partly evaporated fermented fine-divided waste that has a reduced amount of fermentation inhibitors from the vaporization and dehydration vessel to the fermentation vessel for further or additional fermentation of the partly evaporated fermented fine-divided waste, resulting in higher yield and fermentation rate.

Said vaporization and dehydrating means comprises preferably, but not necessarily, mixing means for mixing or moving said fermented fine-divided waste during an evaporation step for at least partly separating by evaporation said mixture of ethanol and water from said fermented fine-divided waste and during a dehydrating step for dehydrating said fermented fine-divided waste to produce dry matter of the fermented fine-divided waste. Said vaporization and dehydrating means can for example comprise a rotary dryer.

In one embodiment of the invention the apparatus comprises a combined hydrolyzing and fermentation and vaporization and dehydration vessel comprising a crusher. In this preferred embodiment the combined hydrolyzing and fermentation and vaporization and dehydration vessel comprising the crusher is preferably sealed in such a way that substantially all vapor separated i.e. vaporized from the fermented fine-divided water is discharged from the combined hydrolyzing and fermentation and vaporization and dehydration vessel through the first discharging means. This preferred embodiment of the invention is especially suitable for small-scale production of ethanol and solid biofuel in the form of dry matter.

In another embodiment of the invention the apparatus comprises a separate crusher, a separate hydrolyzing vessel, a separate fermentation vessel and a separate vaporization and dehydration vessel comprising a rotary dryer. In this preferred embodiment the separate vaporization and dehydration vessel comprising the rotary dryer is preferably sealed in such a way that substantially all vapor separated i.e. vaporized from the fermented fine-divided water containing mixture of ethanol and water is discharged from the separate vaporization and dehydration vessel through the first discharging means and thereafter fed to a concentration means for producing a concentrated mixture of ethanol and water. The vapor is preferably, but not necessarily, filtrated or in a corresponding manner purified prior to feeding the vapor discharged from said first discharging means of said separate vaporization and dehydration vessel to said concentration means. This preferred embodiment of the invention is especially suitable for large-scale production of ethanol and solid biofuel in the form of dry matter.

In a preferred embodiment of the invention the saccharification step is performed without adding water or other liquids to the fine-divided waste, provided that the liquid content of the waste for example the liquid content of the municipal biodegradable waste is sufficient for the saccharification step. In many cases, it is normally possible to produce—with a method according to the invention—a mixture of ethanol and water without adding water or any other liquids to the waste. At least the liquid percentage of municipal biodegradable waste between 70 and 75 has been found to be sufficient and makes additional adding of liquids unnecessary.

Advantages:Possible to produce both liquid biofuel and solid biofuel from waste with one and the same apparatus for example with a method and apparatus described and shown in FIGS. 1 to 8 or even in one apparatus for example with an apparatus shown in FIG. 4.

Dry matter can be used as a solid biofuel for the production of thermal energy to be used in the process or to be used elsewhere. The thermal energy can be utilized in heat exchanges functionally connected to at least one of the hydrolyzing means, the fermentation means and the vaporization and dehydrating means

Process is very simple. For example in the embodiment shown in FIG. 4 all physico-chemical steps (fine-dividing, saccharification (hydrolysis), fermentation, vaporization, and drying) occur in one and the same vessel. On the other hand, the physico-chemical steps (fine-dividing, saccharification (hydrolysis), fermentation, vaporization, and drying) can be divided to occur in separate vessels still keeping the process simple.

With a method and apparatus of the invention a wide range of waste can be processed.

With a method and apparatus of the invention can ethanol be produced from waste containing fermentable sugars or starch and/or cellulose which can be saccharified into fermentable sugars.

### LIST OF FIGURES

In the following the invention will be described in more detail by referring to the figures, of which

FIG. 1 shows a flow sheet of a first preferred embodiment of the invention,

FIG. 2 shows a flow sheet of a second preferred embodiment of the invention,

FIG. 3 shows a flow sheet of a third preferred embodiment of the invention,

FIG. 4 shows a flow sheet of a fourth preferred embodiment of the invention,

FIG. 5 shows a flow sheet of a fifth preferred embodiment of the invention,

FIG. 6 shows a flow sheet of a sixth preferred embodiment of the invention,

FIG. 7 shows a flow sheet of a seventh preferred embodiment of the invention,

FIG. 8 shows a flow sheet of an eighth preferred embodiment of the invention,

FIG. 9 shows a flow sheet of a ninth preferred embodiment of the invention,

FIG. 10 shows a flow sheet of a tenth preferred embodiment of the invention

FIG. 11 shows a flow sheet of a eleventh preferred embodiment of the invention,

FIG. 12 shows a flow sheet of a twelfth preferred embodiment of the invention

FIG. 13 shows a flow sheet of a thirteenth preferred embodiment of the invention,

FIG. 14 shows a flow sheet of a fourteenth preferred embodiment of the invention

FIG. 15 shows a flow sheet of a fifteenth preferred embodiment of the invention, and

FIG. 16 shows a flow sheet of a sixteenth preferred embodiment of the invention.

### DETAILED DESCRIPTION OF THE INVENTION

The invention relates firstly to an apparatus for processing waste 1 containing fermentable raw materials selected from sugars and raw materials such as starch and cellulose capable of being saccharified into fermentable sugars.

The apparatus is preferably, but not necessarily, configured for processing municipal biodegradable waste 1 containing fermentable raw materials selected from sugars and raw materials such as starch and cellulose capable of being saccharified into fermentable sugars.

The apparatus comprises crushing means 2 for at least partly fine-dividing said waste 1 containing fermentable raw materials selected from sugars and raw materials such as starch and cellulose capable of being saccharified into fermentable sugars to form fine-divided waste 30.

The apparatus can in addition comprise sorting means (not shown in the figures) for removing possible non-fermentable matter such as plastics and bones present in the waste 1 before feeding said waste 1 to said crushing means 2.

Said crushing means 2 is preferably, but not necessarily, configured to also fine-divide possible non-fermentable matter such as plastics and bones present in the waste 1. The preferred particle size in the fine-divided waste 30 is 0.5 to 2 mm.

The apparatus can in addition comprise screening means (not shown in the figures) for screening said fine-divided waste 30 to remove possible non-fermentable matter such as plastics and bones present in the fine-divided waste 30 after fine-dividing said waste.

The apparatus comprises hydrolyzing means 3 for at least partly saccharifying with saccharifying enzymes said fine-divided waste 30 to form saccharified fine-divided waste 31. The saccharifying enzymes break down at least raw materials such as starch and cellulose capable of being saccharified into fermentable sugars present in the fine-divided waste 30 to produce fermentable sugars.

The apparatus comprises fermentation means 4 for at least partly fermenting with ethanol producing microorganisms 23 such as yeast said saccharified fine-divided waste 31 to form fermented fine-divided waste 32 containing a mixture of ethanol and water 25. The micro-organisms such as yeast use fermentable sugars in the saccharified fine-divided waste to produce ethanol i.e. to produce said fermented fine-divided waste 32 containing a mixture of ethanol and water 25.

The apparatus comprises vaporization and dehydrating means 5 for at least partly separating by vaporization said mixture of ethanol and water 25 from said fermented fine-divided waste 32 and for dehydrating said fermented fine-divided waste 32 in order to form at least partly dry matter 24 of said fermented fine-divided waste 32.

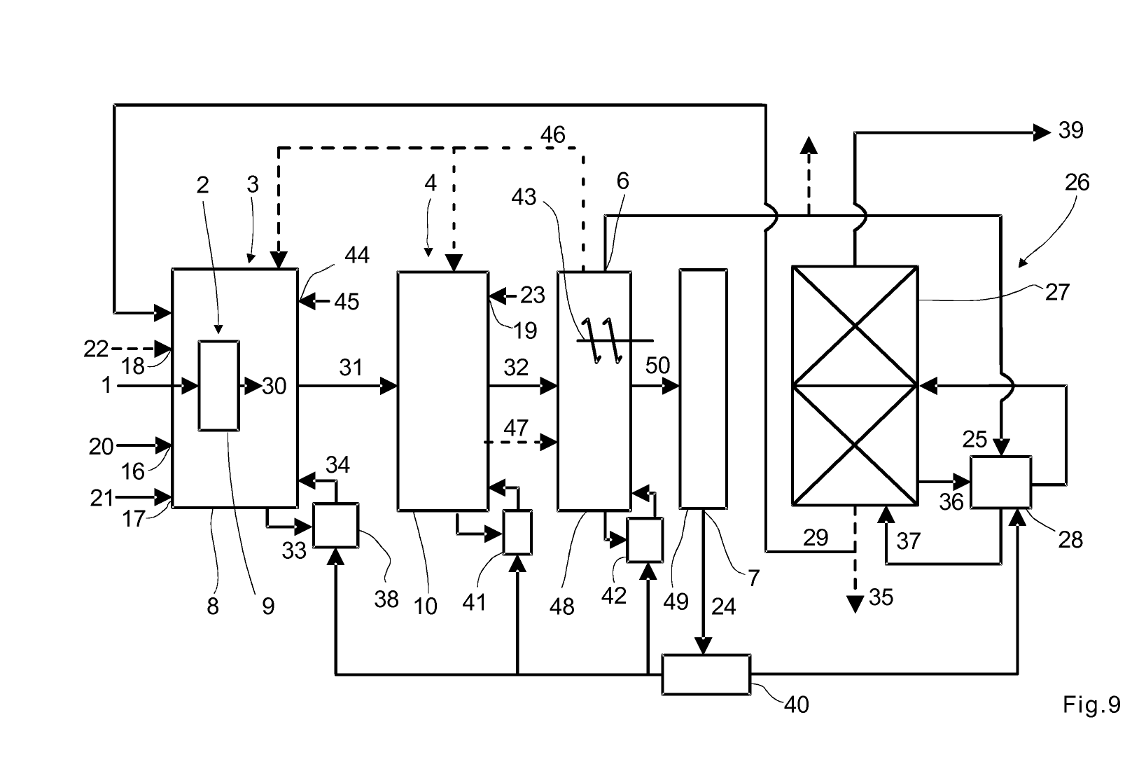

Said vaporization and dehydrating means 5 can be divided into a separate vaporization means 51 for at least partly separating by vaporization said mixture of ethanol and water 25 in said fermented fine-divided waste 32 and a separate dehydrating means 52 for dehydrating fermented fine-divided waste 32 after the fermented fine-divided waste 32 has been treated in said separate vaporization means 51 for at least partly separating by vaporization said mixture of ethanol and water 25 from said fermented fine-divided waste 32. Such embodiments are shown in FIGS. 9 to 12.

Said vaporization and dehydrating means 5 comprises preferably mixing means 43 for mixing or otherwise moving said fermented fine-divided waste 32 in said vaporization and dehydrating means 5.

The vaporization and dehydrating means 5 comprises preferably a drum dryer or a flash dryer.

The vaporization and dehydrating means 5 can for example comprise a dryer apparatus of the type disclosed in publication WO/2006/024696.

The apparatus comprises first discharging means 6 for discharging said vaporized mixture of ethanol and water 25 from said vaporization and dehydrating means 5.

The apparatus comprises second discharging means 7 for discharging said dry matter 24 from said vaporization and dehydrating means 5. The apparatus may comprise conduit means (not marked with a reference numeral) for feeding partly dehydrated fermented fine-divided waste 46 from said vaporization and dehydrating means 5 back to at least of the following: said crushing means 2, said hydrolyzing means 3, and said fermentation means 4.

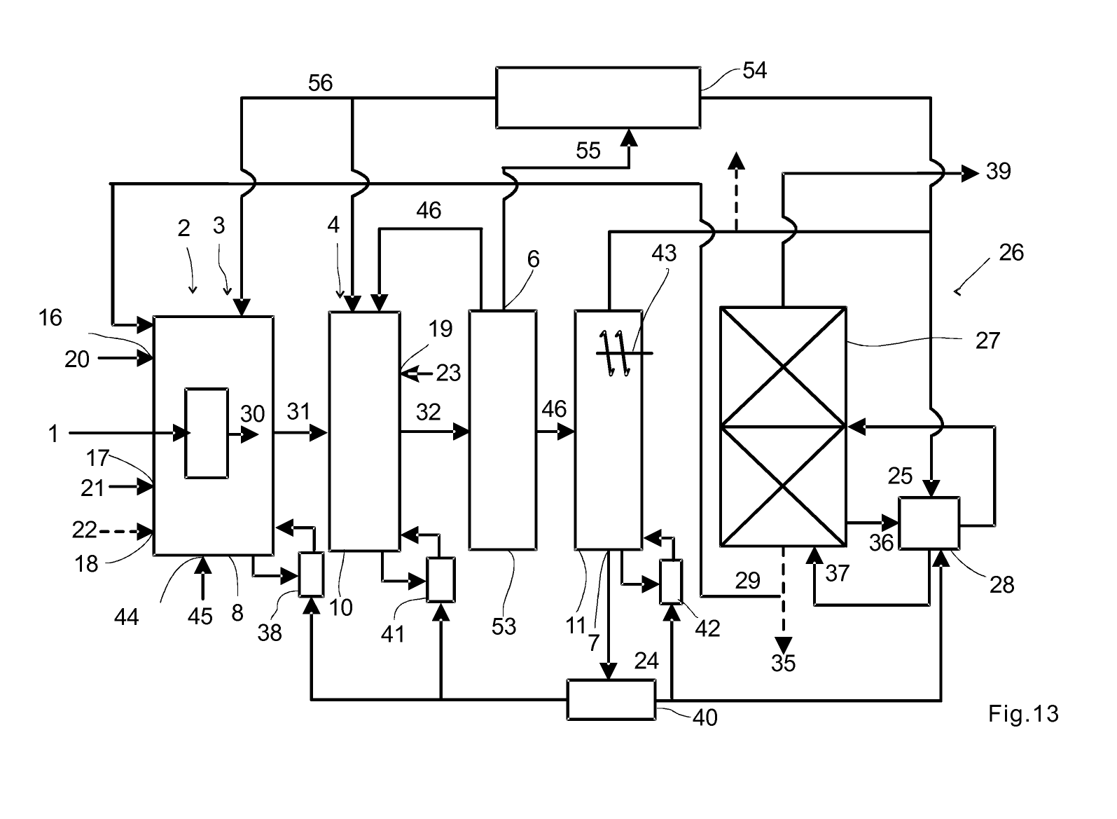

The apparatus may comprise a solid matter separator means 53 that is arranged downstream of the fermentation means 4 and upstream of the vaporization and dehydration means 5. The apparatuses illustrated in FIGS. 13 to 16 comprise a solid matter separator means 53. The solid matter separator means 53 is configured for separating fermentation beer 55 or ethanol beer from fermented fine divided waste 32 that is fed into the solid matter separator means 53, in other words, the solid matter separator means 53 is configured for separating fermentation beer 55 or ethanol beer from fermented fine divided waste 32 that is fed into the solid matter separator means 53 so that the result is fermentation beer 55 and partly dehydrated fermented fine-divided waste 46. The solid matter separation means 53 can be a mechanical press such as a screw press for mechanically pressing fermentation beer 55 out of fermented fine divided waste 32. If the apparatus comprises a solid matter separator means 53, the apparatus preferably also comprises an evaporator means 54, as shown in FIGS. 13 to 16, that is in fluid connection with the solid matter separator means 53 and that is configured for receiving fermentation beer 55 from the solid matter separator means 53. If the apparatus comprises a solid matter separator means 53 the solid matter separator means 53 is preferably functionally connected with the vaporization and dehydration means 5 so that the vaporization and dehydration means 5 is configured for receiving partly dehydrated fermented fine-divided waste 46 from the solid matter separator means 53. The evaporator means 54 comprises preferably a falling film evaporator that is configured to divide fermentation beer 55 into mixture of ethanol and water 25 and into a residual bottom stream 56. If the apparatus comprises an evaporator means 54, the evaporator means 54 is preferably configured for feeding the residual bottom stream 56 to at least one of the crushing means 2, the hydrolyzing means 3, or the fermentation means 4.

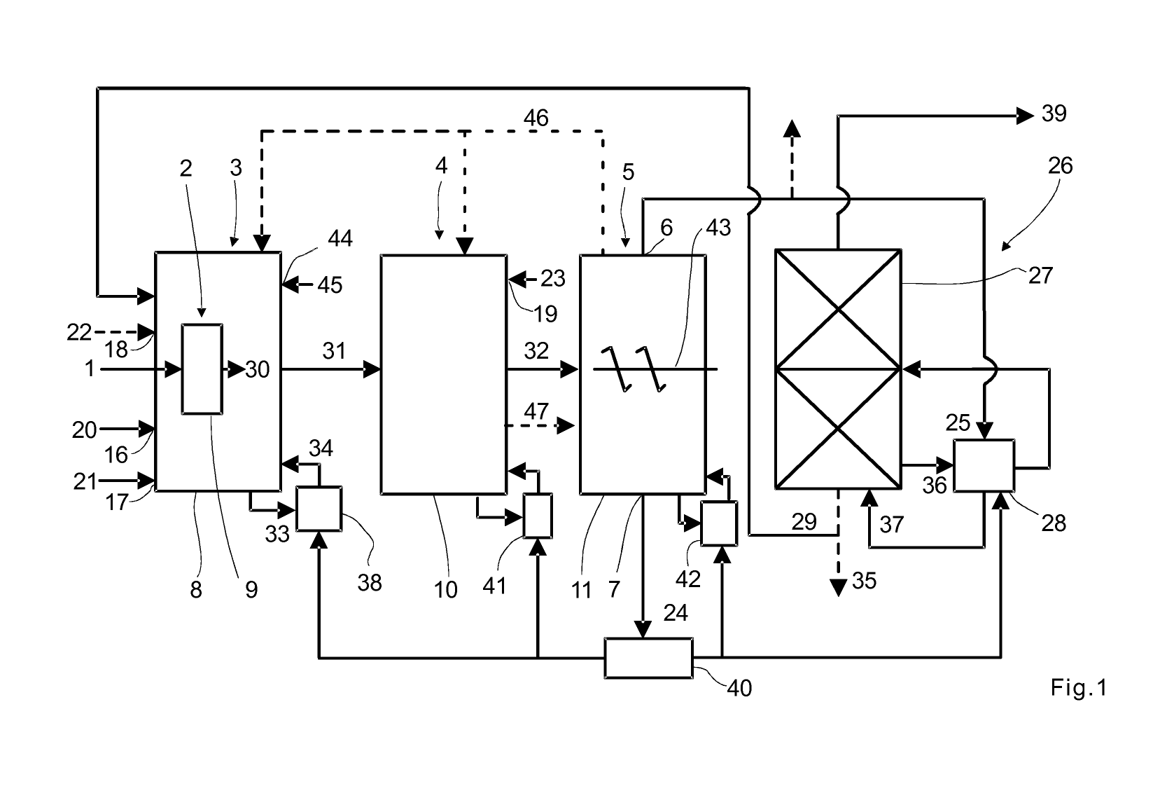

Said hydrolyzing means 3 comprises in a first preferred embodiment of the apparatus of the invention shown in FIG. 1, a separate hydrolyzing vessel 8 for saccharifying with saccharifying enzymes said fine-divided waste 30 to form saccharified fine-divided waste 31.

In this first preferred embodiment of the invention said crushing means 2 is a crusher 9 arranged in said separate hydrolyzing vessel 8. In this preferred embodiment of the invention said fermentation means 4 comprises a separate fermentation vessel 10 for fermenting with ethanol producing microorganisms 23 such as yeast said saccharified fine-divided waste 31 to form fermented fine-divided waste 32 containing a mixture of ethanol and water 25.

In this first preferred embodiment of the invention said vaporization and dehydrating means 5 comprises a separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32. The separate vaporization and dehydration vessel 11 may, as shown in the ninth embodiment shown in FIG. 9, that otherwise corresponds to the first embodiment shown in FIG. 1, be divided into a separate vaporization vessel 48 for at least partly separating by vaporization said mixture of ethanol and water 25 from said fermented fine-divided waste 32 and into separate dehydration vessel 49 for dehydrating said fermented fine-divided waste 32 to form dry matter 24 of said fermented fine-divided waste 32.

The apparatus shown in FIG. 1 comprises conduit means (not marked with a reference numeral) for feeding saccharified fine-divided waste 31 from said separate hydrolyzing vessel 8 to said separate fermentation vessel 10.

The apparatus shown in FIG. 1 comprises in addition conduit means (not marked with a reference numeral) for feeding fermented fine-divided waste 32 containing a mixture of ethanol and water 25 from said separate fermentation vessel 10 to said separate vaporization and dehydration vessel 11.

The apparatus shown in FIG. 1 comprises in addition conduit means (not marked with a reference numeral) for feeding partly dehydrated fermented fine-divided waste 46 from said separate vaporization and dehydration vessel 11 to said separate hydrolyzing vessel 8 and to said separate fermentation vessel 10. The apparatus shown in FIG. 1 comprises more preferably conduit means (not marked with a reference numeral) for feeding partly dehydrated fermented fine-divided waste 46 from said separate vaporization and dehydration vessel 11 only to said separate fermentation vessel 10. The apparatus shown in FIG. 1 comprises in addition conduit means (not marked with a reference numeral) for feeding fermented fine-divided waste containing fermentation inhibitors 47 from said separate fermentation vessel 10 to said separate vaporization and dehydration vessel 11 so that fermentation inhibitors can be evaporated off the fermented fine-divided waste containing fermentation inhibitors 47 in said separate vaporization and dehydration vessel 11 so that partly dehydrated fermented fine-divided waste 46 is obtained that can be fed back to said separate hydrolyzing vessel 8 and/or to said separate fermentation vessel 10.

The apparatus may as in the thirteenth embodiment shown in FIG. 13 that otherwise essentially corresponds to the first embodiment shown in FIG. 1, comprise a solid matter separator means 53 that is arranged downstream of the separate fermentation vessel 10 and upstream of the separate vaporization and dehydration vessel 11 and a evaporation means 54 that is in fluid connection with the solid matter separation means 53. In FIG. 13 the solid matter separator means 53 is configured for separating fermentation beer 55 or ethanol beer from fermented fine divided waste 32 that is fed into the solid matter separator means 53 from the separate fermentation vessel 10. In FIG. 13 the solid matter separator means 53 is in fluid connection with the evaporation means 54 that is configured for receiving fermentation beer 55 from the solid matter separator means 53. In FIG. 13 the solid matter separator means 53 is also connected with the separate vaporization and dehydration vessel 10 so that the separate vaporization and dehydration vessel 10 is configured for receiving partly dehydrated fermented fine-divided waste 46 from the solid matter separator means 53. In FIG. 13 the solid matter separator means 53 is also connected with the separate fermentation vessel 10 so that partly dehydrated fermented fine-divided waste 46 can be fed from the solid matter separator means 53 to the separate fermentation vessel 10. The evaporator means 54 comprises preferably a falling film evaporator that is configured to divide fermentation beer 55 into mixture of ethanol and water 25 and into a residual bottom stream 56. The residual bottom stream 56 can for example contain ethanol producing microbes and sugars that can be re-used in the process. Therefore it is advantageous to use a falling-film evaporator in the evaporator means 54 because then the evaporation can be performed at a such temperature and pressure which does not kill possible ethanol producing microbes that can be re-used in the process. In FIG. 13 the evaporator means 54 is configured for feeding the residual bottom stream 56 to the hydrolyzing means 3 and the fermentation means 4. In FIG. 13, the evaporator means 54 is configured for feeding mixture of ethanol and water 25 to a concentration means 26.

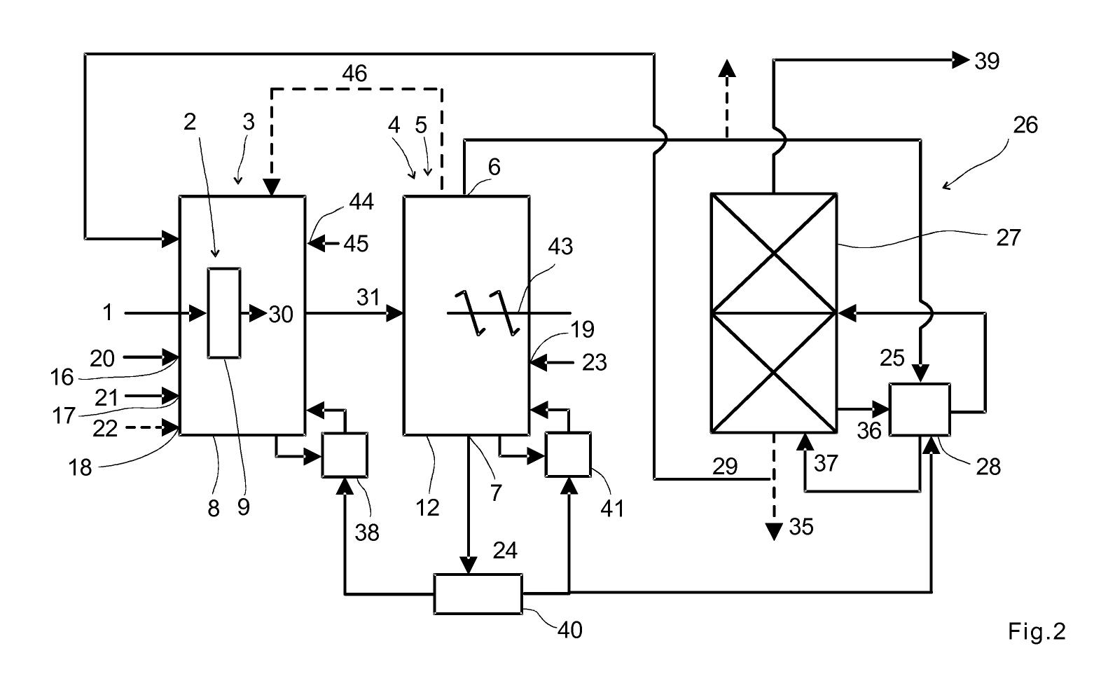

The apparatus comprises in a second preferred embodiment of the apparatus of the invention shown in FIG. 2 a separate hydrolyzing vessel 8 for saccharifying with saccharifying enzymes said fine-divided waste 30 to form saccharified fine-divided waste 31.

In this second preferred embodiment of the invention said crushing means 2 is a crusher 9 arranged in said separate hydrolyzing vessel 8.

Said apparatus comprises in FIG. 2 a combined fermentation and vaporization and dehydration vessel 12 for firstly fermenting with ethanol-producing microorganisms 23 such as yeast or fermenting microorganisms said saccharified fine-divided waste 31 to form fermented fine-divided waste 32 containing a mixture of ethanol and water 25 in said combined hydrolyzing and fermentation and vaporization and dehydration vessel 14. Said combined fermentation and vaporization and dehydration vessel 12 in FIG. 2 is secondly configured for at least partly separating by vaporization said mixture of ethanol and water 25 from said fermented fine-divided waste 32 in said combined hydrolyzing and fermentation and vaporization and dehydration vessel 14 and for dehydrating said fermented fine-divided waste 32 to form dry matter 24 of said fermented fine-divided waste 32 in said combined hydrolyzing and fermentation and vaporization and dehydration vessel 14.

In this second preferred embodiment of the invention shown in FIG. 2 said crushing means 2 is a crusher 9 arranged in said combined hydrolyzing and fermentation and vaporization and dehydration vessel 14.

The apparatus shown in FIG. 2 comprises conduit means (not marked with a reference numeral) for feeding saccharified fine-divided waste 31 from said separate hydrolyzing vessel 8 to said combined fermentation and vaporization and dehydration vessel 12.

The apparatus shown in FIG. 2 comprises in addition conduit means (not marked with a reference numeral) for feeding partly dehydrated fermented fine-divided waste 46 from said combined fermentation and vaporization and dehydration vessel 12 to said separate hydrolyzing vessel 8. The apparatus shown in FIG. 2 does more preferably not comprise any conduit means for feeding partly dehydrated fermented fine-divided waste 46 from said separate combined fermentation and vaporization and dehydration vessel 12 to said separate hydrolyzing vessel 8.

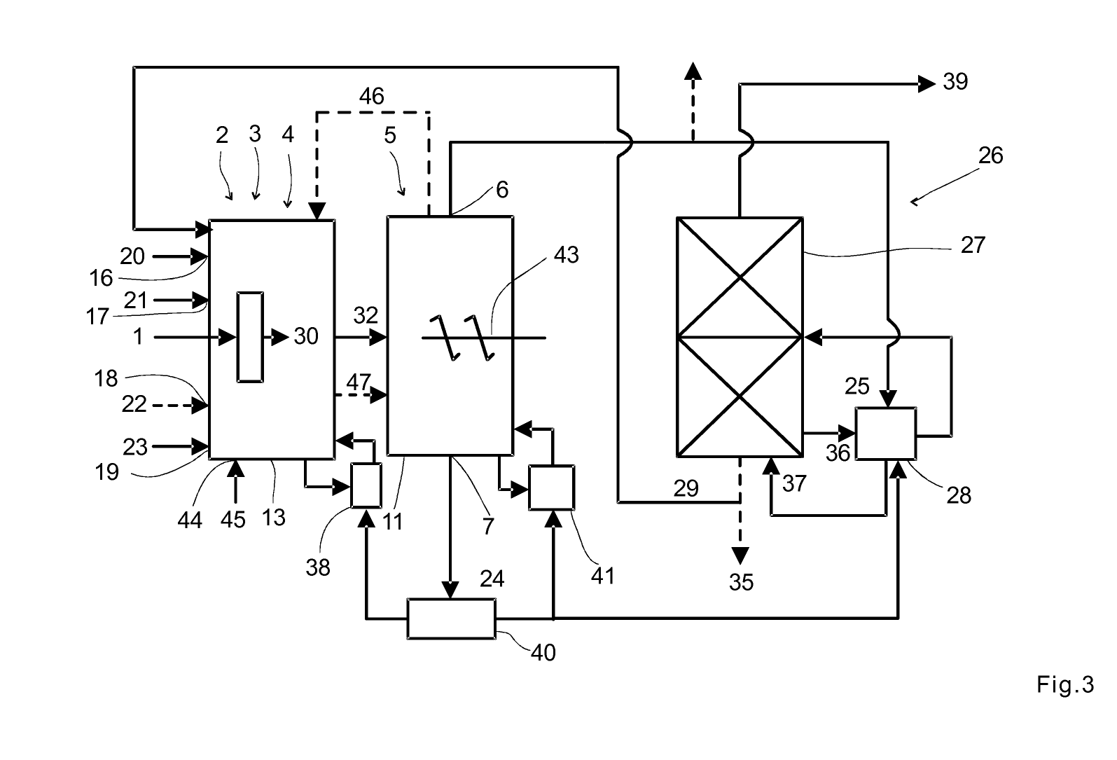

Said hydrolyzing means 3 and said fermentation means 4 comprises in a third preferred embodiment of the apparatus of the invention shown in FIG. 3 a combined hydrolyzing and fermentation vessel 13 for firstly saccharifying with saccharifying enzymes said fine-divided waste 30 to form saccharified fine-divided waste 31 and thereafter for fermenting with ethanol-producing microorganisms 23 such as yeast or fermenting microorganisms said saccharified fine-divided waste 31 to form fermented fine-divided waste 32 containing a mixture of ethanol and water 25.

In this third preferred embodiment of the invention said crushing means 2 is a crusher 9 arranged in said combined hydrolyzing and fermentation vessel 13.

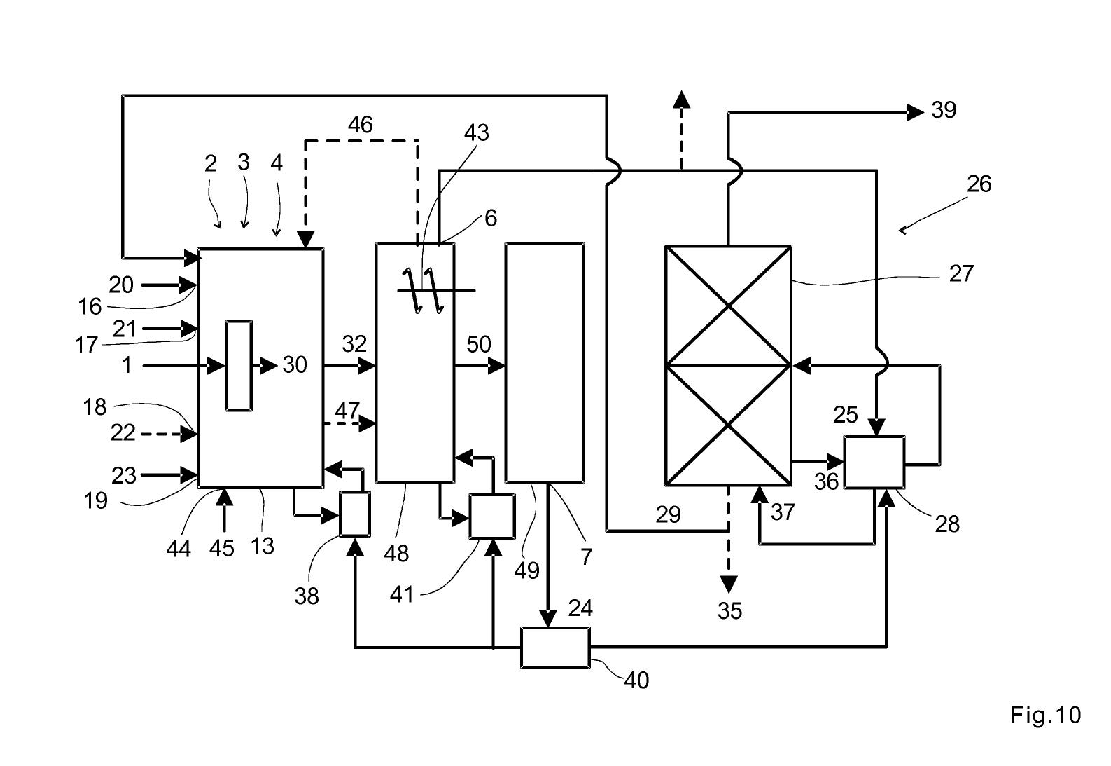

Said vaporization and dehydrating means 5 comprises in this third preferred embodiment of the invention a separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32. The separate vaporization and dehydration vessel 11 may, as shown in the tenth embodiment shown in FIG. 10, that otherwise corresponds to the third embodiment shown in FIG. 3, be divided into a separate vaporization vessel 48 for at least partly separating by vaporization said mixture of ethanol and water 25 from said fermented fine-divided waste 32 and into separate dehydration vessel 49 for dehydrating said fermented fine-divided waste 32 to form dry matter 24 of said fermented fine-divided waste 32.

The apparatus shown in FIG. 3 comprises in addition conduit means (not marked with a reference numeral) for feeding fermented fine-divided waste 32 containing mixture of ethanol and water 25 from said combined hydrolyzing and fermentation vessel 13 to said separate vaporization and dehydration vessel 11.

The apparatus shown in FIG. 3 comprises in addition conduit means (not marked with a reference numeral) for feeding partly dehydrated fermented fine-divided waste 46 from said separate vaporization and dehydration vessel 11 to said combined hydrolyzing and fermentation vessel 13. The apparatus shown in FIG. 3 comprises in addition conduit means (not marked with a reference numeral) for feeding fermented fine-divided waste containing fermentation inhibitors 47 from said combined hydrolyzing and fermentation vessel 13 to said separate vaporization and dehydration vessel 11 so that fermentation inhibitors can be evaporated off the fermented fine-divided waste containing fermentation inhibitors 47 in said separate vaporization and dehydration vessel 11 so that partly dehydrated fermented fine-divided waste 46 is obtained that can be fed back to said combined hydrolyzing and fermentation vessel 13.

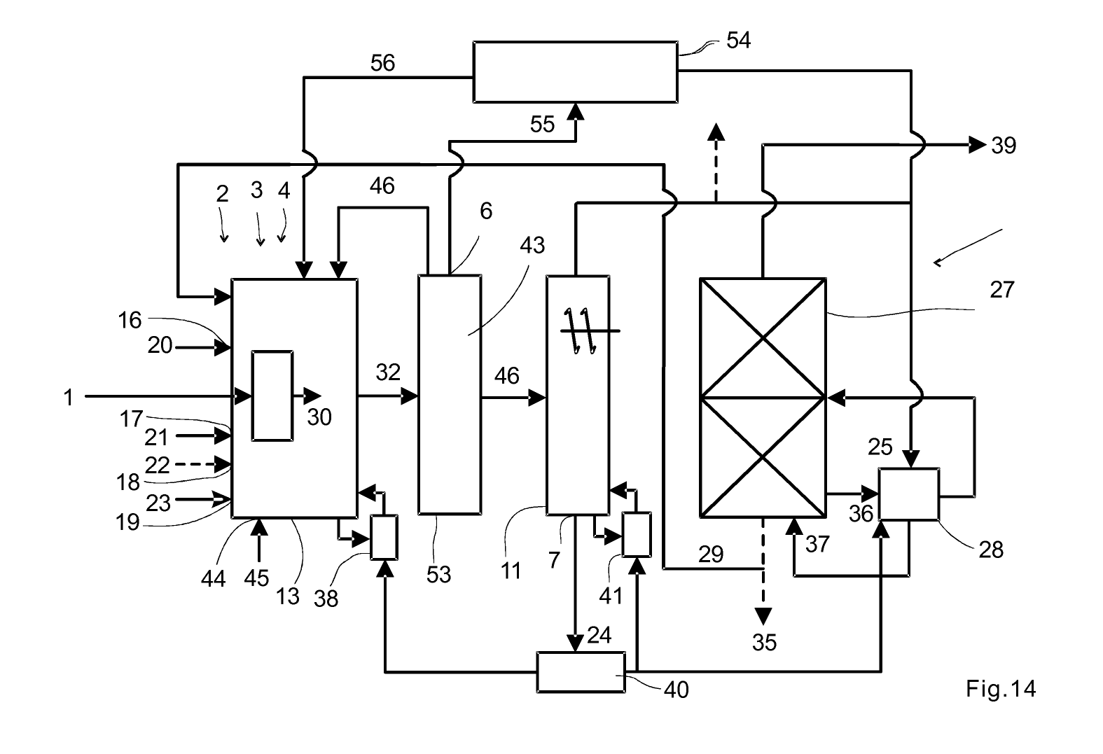

The apparatus may as in the fourteenth embodiment shown in FIG. 14 that otherwise essentially corresponds to the third embodiment shown in FIG. 3, comprise a solid matter separator means 53 that is arranged downstream of the combined hydrolyzing and fermentation vessel 13 and upstream of the separate vaporization and dehydration vessel 11 and a evaporation means 54 that is in fluid connection with the solid matter separation means 53. In FIG. 14 the solid matter separator means 53 is configured for separating fermentation beer 55 or ethanol beer from fermented fine divided waste 32 that is fed into the solid matter separator means 53 from the combined hydrolyzing and fermentation vessel 13. In FIG. 14 the solid matter separator means 53 is in fluid connection with the evaporation means 54 that is configured for receiving fermentation beer 55 from the solid matter separator means 53. In FIG. 14 the solid matter separator means 53 is also connected with the vaporization and dehydration means 5 so that the separate vaporization and dehydration vessel 11 is configured for receiving partly dehydrated fermented fine-divided waste 46 from the solid matter separator means 53. In FIG. 14 the solid matter separator means 53 is also connected with the combined hydrolyzing and fermentation vessel 13 so that partly dehydrated fermented fine-divided waste 46 can be fed from the solid matter separator means 53 to the separate fermentation vessel 10. The evaporator means 54 comprises preferably a falling film evaporator that is configured to divide fermentation beer 55 into mixture of ethanol and water 25 and into a residual bottom stream 56. The residual bottom stream 56 can for example contain ethanol producing microbes and sugars that can be re-used in the process. Therefore it is advantageous to use a falling-film evaporator in the evaporator means 54 because then the evaporation can be performed at a such temperature and pressure which does not kill possible ethanol producing microbes that can be re-used in the process. In FIG. 14 the evaporator means 54 is configured for feeding the residual bottom stream 56 to the combined hydrolyzing and fermentation vessel 13. In FIG. 14, the evaporator means 54 is configured for feeding mixture of ethanol and water 25 to a concentration means 26.

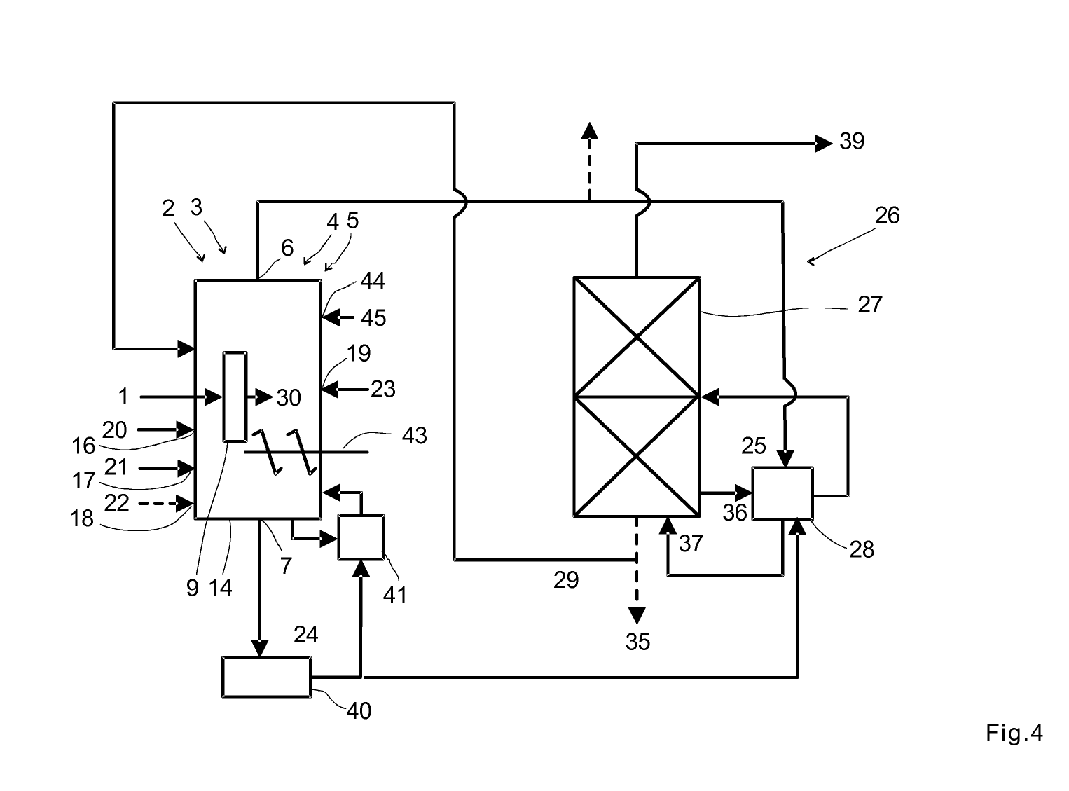

Said hydrolyzing means 3 and said fermentation means 4 and said vaporization and dehydrating means 5 comprise in a fourth preferred embodiment of the apparatus of the invention shown in FIG. 4 a combined hydrolyzing and fermentation and vaporization and dehydration vessel 14 for firstly saccharifying with saccharifying enzymes said fine-divided waste 30 to form saccharified fine-divided waste 31 and thereafter for fermenting with ethanol-producing microorganisms 23 such as yeast or fermenting microorganisms said saccharified fine-divided waste 31 to form fermented fine-divided waste 32 containing mixture of ethanol and water 25 and thereafter for at least partly separating by vaporization said mixture of ethanol and water 25 from said fermented fine-divided waste 32 and for dehydrating said fermented fine-divided waste 32 to form dry matter 24 of said fermented fine-divided waste 32.

In this fourth preferred embodiment of the invention said crushing means 2 is a crusher 9 arranged in said combined hydrolyzing and fermentation and vaporization and dehydration vessel 14.

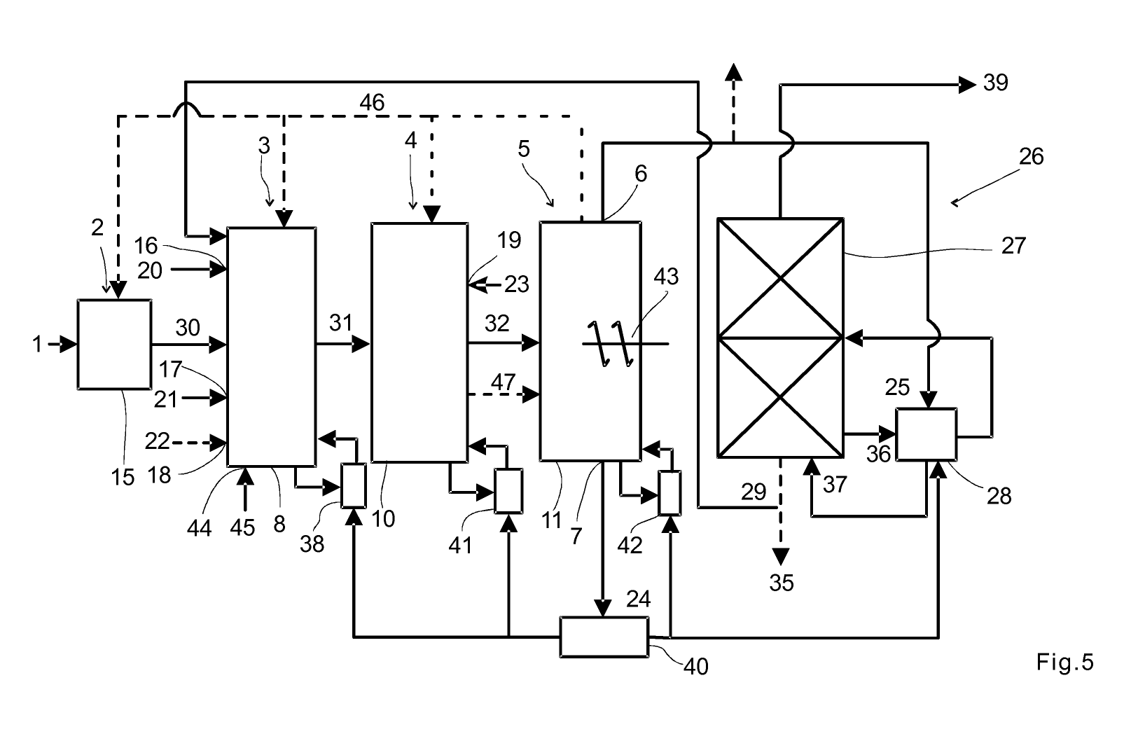

In a fifth preferred embodiment of the apparatus of the invention shown in FIG. 5 said crushing means 2 comprises a separate crusher 15 for crushing waste 1 to form fine-divided waste 30 of the waste 1.

In this fifth preferred embodiment of the invention said hydrolyzing means 3 comprises a separate hydrolyzing vessel 8 saccharifying with saccharifying enzymes said fine-divided waste 30 to form saccharified fine-divided waste 31.

In this fifth preferred embodiment of the invention said fermentation means 4 comprises a separate fermentation vessel 10 for fermenting with ethanol-producing microorganisms 23 such as yeast or fermenting microorganisms said saccharified fine-divided waste 31 to form fermented fine-divided waste 32 containing a mixture of ethanol and water 25.

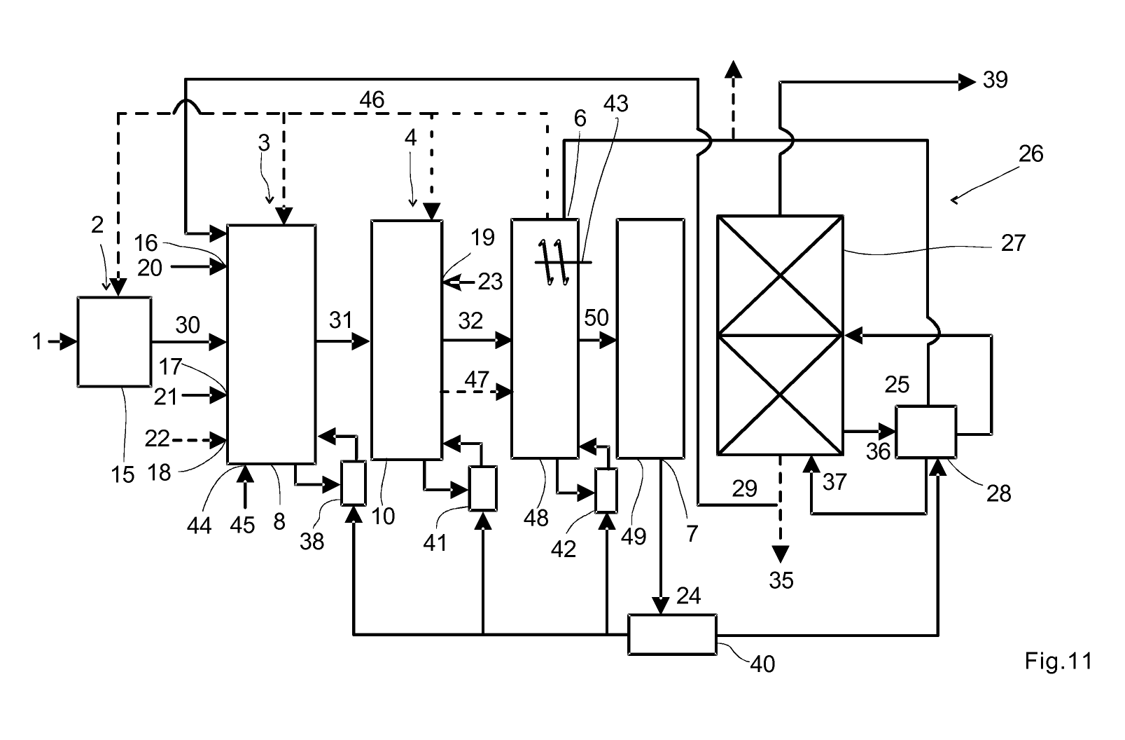

In this fifth preferred embodiment of the invention said vaporization and dehydrating means 5 comprises a separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32. The separate vaporization and dehydration vessel 11 may, as shown in the eleventh embodiment shown in FIG. 11, that otherwise corresponds to the fifth embodiment shown in FIG. 5, be divided into a separate vaporization vessel 48 for at least partly separating by vaporization said mixture of ethanol and water 25 from said fermented fine-divided waste 32 and into separate dehydration vessel 49 for dehydrating said fermented fine-divided waste 32 to form dry matter 24 of said fermented fine-divided waste 32.

The apparatus shown in FIG. 5 comprises conduit means (not marked with a reference numeral) for feeding fine-divided waste 30 from said separate crusher 15 to said separate hydrolyzing vessel 8.

The apparatus shown in FIG. 5 comprises in addition conduit means (not marked with a reference numeral) for feeding saccharified fine-divided waste 31 from said separate hydrolyzing vessel 8 to said separate fermentation vessel 10.

The apparatus shown in FIG. 5. comprises in addition conduit means (not marked with a reference numeral) for feeding fermented fine-divided waste 32 containing mixture of ethanol and water 25 from said separate fermentation vessel 10 to said separate vaporization and dehydration vessel 11.

The apparatus shown in FIG. 5 comprises in addition conduit means (not marked with a reference numeral) for feeding partly dehydrated fermented fine-divided waste 46 from said separate vaporization and dehydration vessel 11 to said separate crusher 15, to said separate hydrolyzing vessel 8 and to said separate fermentation vessel 10. The apparatus shown in FIG. 5 comprises more preferably conduit means (not marked with a reference numeral) for feeding partly dehydrated fermented fine-divided waste 46 from said separate vaporization and dehydration vessel 11 only to said separate fermentation vessel 10. The apparatus shown in FIG. 5 comprises in addition conduit means (not marked with a reference numeral) for feeding fermented fine-divided waste containing fermentation inhibitors 47 from said separate fermentation vessel 10 to said separate vaporization and dehydration vessel 11 so that fermentation inhibitors can be evaporated off the fermented fine-divided waste containing fermentation inhibitors 47 in said separate vaporization and dehydration vessel 11 so that partly dehydrated fermented fine-divided waste 46 is obtained that can be fed back to said separate hydrolyzing vessel 8 and/or to said separate fermentation vessel 10 and/or said separate crusher 15.

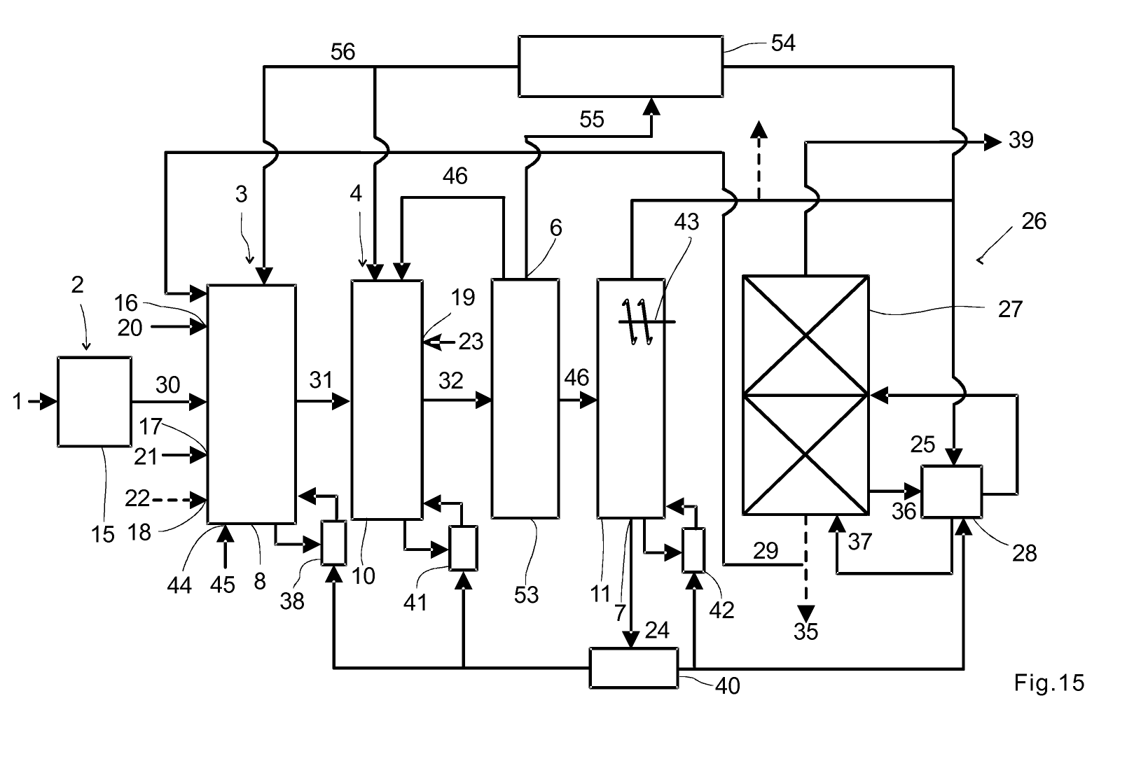

The apparatus may as in the fifteenth embodiment shown in FIG. 15 that otherwise essentially corresponds to the fifth embodiment shown in FIG. 5, comprise a solid matter separator means 53 that is arranged downstream of the separate fermentation vessel 10 and upstream of the separate vaporization and dehydration vessel 11 and a evaporation means 54 that is in fluid connection with the solid matter separation means 53. In FIG. 15 the solid matter separator means 53 is configured for separating fermentation beer 55 or ethanol beer from fermented fine divided waste 32 that is fed into the solid matter separator means 53 from the separate fermentation vessel 10. In FIG. 15 the solid matter separator means 53 is in fluid connection with the evaporation means 54 that is configured for receiving fermentation beer 55 from the solid matter separator means 53. In FIG. 15 the solid matter separator means 53 is also connected with the separate vaporization and dehydration vessel 10 so that the separate vaporization and dehydration vessel 10 is configured for receiving partly dehydrated fermented fine-divided waste 46 from the solid matter separator means 53. In FIG. 15 the solid matter separator means 53 is also connected with the separate fermentation vessel 10 so that partly dehydrated fermented fine-divided waste 46 can be fed from the solid matter separator means 53 to the separate fermentation vessel 10. The evaporator means 54 comprises preferably a falling film evaporator that is configured to divide fermentation beer 55 into mixture of ethanol and water 25 and into a residual bottom stream 56. The residual bottom stream 56 can for example contain ethanol producing microbes and sugars that can be re-used in the process. Therefore it is advantageous to use a falling-film evaporator in the evaporator means 54 because then the evaporation can be performed at a such temperature and pressure which does not kill possible ethanol producing microbes that can be re-used in the process. In FIG. 15 the evaporator means 54 is configured for feeding the residual bottom stream 56 to the hydrolyzing means 3 and the fermentation means 4. In FIG. 15, the evaporator means 54 is configured for feeding mixture of ethanol and water 25 to a concentration means 26.

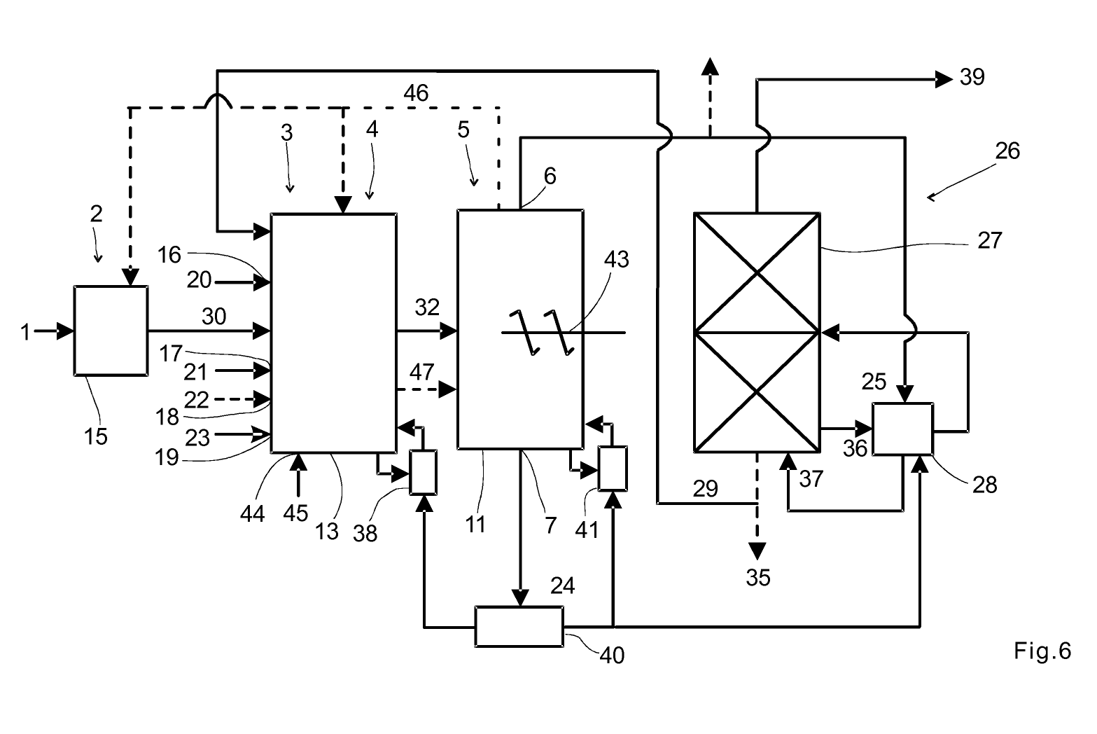

In a sixth preferred embodiment of the apparatus of the invention shown in FIG. 6 said crushing means 2 comprises a separate crusher 15 for crushing waste 1 to form fine-divided waste 30 of the waste 1.

In this sixth preferred embodiment of the invention said hydrolyzing means 3 and said fermentation means 4 comprises a combined hydrolyzing and fermentation vessel 13 for firstly saccharifying with saccharifying enzymes said fine-divided waste to form saccharified fine-divided waste 31 and thereafter for fermenting with ethanol-producing microorganisms 23 such as yeast or fermenting microorganisms said saccharified fine-divided waste 31 to form fermented fine-divided waste 32 containing mixture of ethanol and water 25.

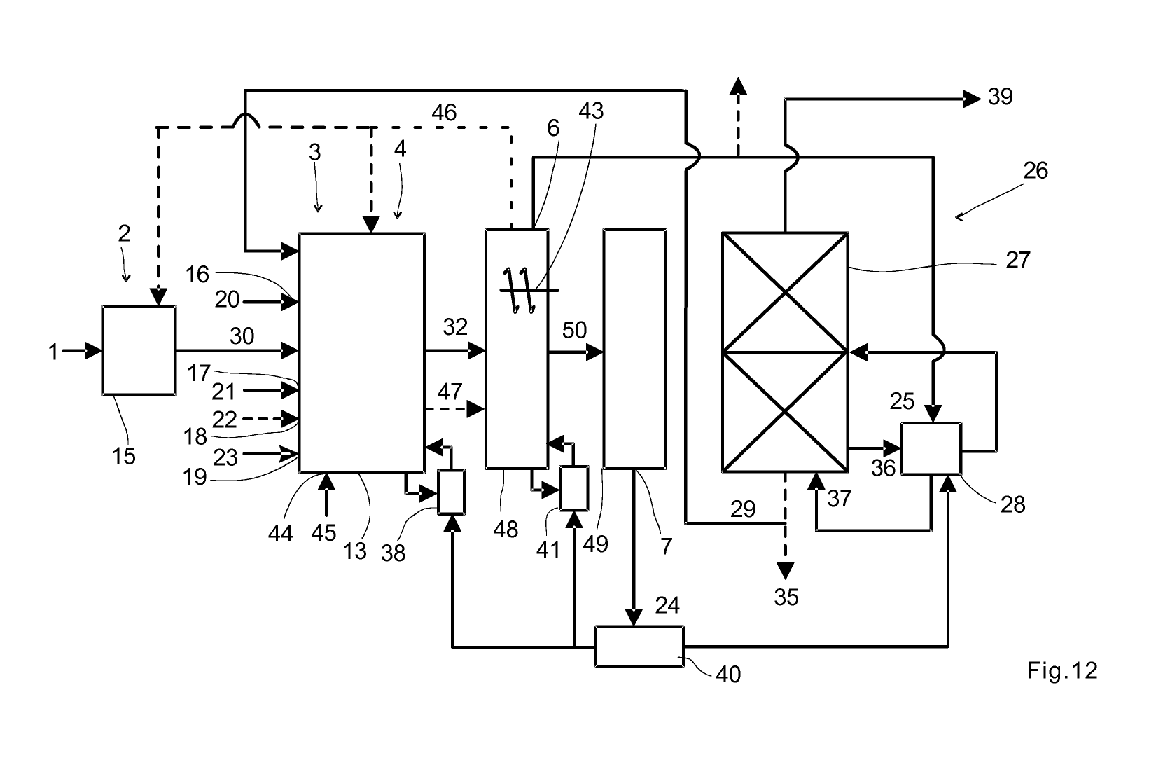

In this sixth preferred embodiment of the invention said vaporization and dehydrating means 5 comprises a separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32. The separate vaporization and dehydration vessel 11 may, as shown in the twelfth embodiment shown in FIG. 12, that otherwise corresponds to the sixth embodiment shown in FIG. 6, be divided into a separate vaporization vessel 48 for at least partly separating by vaporization said mixture of ethanol and water 25 from said fermented fine-divided waste 32 and into separate dehydration vessel 49 for dehydrating said fermented fine-divided waste 32 to form dry matter 24 of said fermented fine-divided waste 32.

The apparatus shown in FIG. 6 comprises conduit means (not marked with a reference numeral) for feeding fine-divided waste 30 from said separate crusher 15 to said separate hydrolyzing vessel 8.

The apparatus shown in FIG. 6 comprises in addition conduit means (not marked with a reference numeral) for feeding fermented fine-divided waste 32 containing mixture of ethanol and water 25 from said combined hydrolyzing and fermentation vessel 13 to said separate vaporization and dehydration vessel 11.

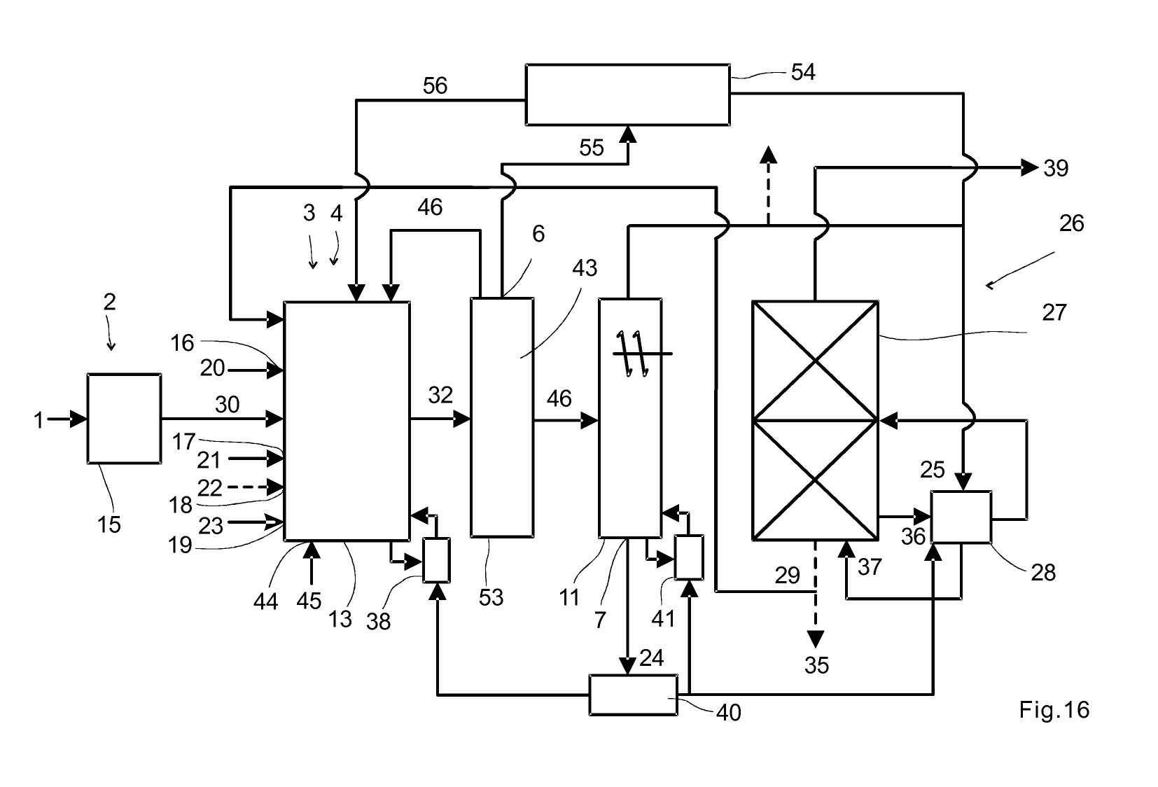

The apparatus shown in FIG. 6 comprises in addition conduit means (not marked with a reference numeral) for feeding partly dehydrated fermented fine-divided waste 46 from said separate vaporization and dehydration vessel 11 to said separate crusher 15 and to said combined hydrolyzing and fermentation vessel 13. The apparatus shown in FIG. 6 comprises more preferably conduit means (not marked with a reference numeral) for feeding partly dehydrated fermented fine-divided waste 46 from said separate vaporization and dehydration vessel 11 only to said combined hydrolyzing and fermentation vessel 13. The apparatus shown in FIG. 6 comprises in addition conduit means (not marked with a reference numeral) for feeding fermented fine-divided waste containing fermentation inhibitors 47 from said combined hydrolyzing and fermentation vessel 13 to said separate vaporization and dehydration vessel 11 so that fermentation inhibitors can be evaporated off the fermented fine-divided waste containing fermentation inhibitors 47 in said separate vaporization and dehydration vessel 11 so that partly dehydrated fermented fine-divided waste 46 is obtained that can be fed back to said combined hydrolyzing and fermentation vessel 13 and/or said separate crusher 15. The apparatus may as in the sixteenth embodiment shown in FIG. 16 that otherwise essentially corresponds to the sixth embodiment shown in FIG. 6, comprise a solid matter separator means 53 that is arranged downstream of the combined hydrolyzing and fermentation vessel 13 and upstream of the separate vaporization and dehydration vessel 11 and a evaporation means 54 that is in fluid connection with the solid matter separation means 53. In FIG. 16 the solid matter separator means 53 is configured for separating fermentation beer 55 or ethanol beer from fermented fine divided waste 32 that is fed into the solid matter separator means 53 from the combined hydrolyzing and fermentation vessel 13. In FIG. 16 the solid matter separator means 53 is in fluid connection with the evaporation means 54 that is configured for receiving fermentation beer 55 from the solid matter separator means 53. In FIG. 16 the solid matter separator means 53 is also connected with the vaporization and dehydration means 5 so that the separate vaporization and dehydration vessel 11 is configured for receiving partly dehydrated fermented fine-divided waste 46 from the solid matter separator means 53. In FIG. 16 the solid matter separator means 53 is also connected with the combined hydrolyzing and fermentation vessel 13 so that partly dehydrated fermented fine-divided waste 46 can be fed from the solid matter separator means 53 to the separate fermentation vessel 10. The evaporator means 54 comprises preferably a falling film evaporator that is configured to divide fermentation beer 55 into mixture of ethanol and water 25 and into a residual bottom stream 56. The residual bottom stream 56 can for example contain ethanol producing microbes and sugars that can be re-used in the process. Therefore it is advantageous to use a falling-film evaporator in the evaporator means 54 because then the evaporation can be performed at a such temperature and pressure which does not kill possible ethanol producing microbes that can be re-used in the process. In FIG. 16 the evaporator means 54 is configured for feeding the residual bottom stream 56 to the combined hydrolyzing and fermentation vessel 13. In FIG. 16, the evaporator means 54 is configured for feeding mixture of ethanol and water 25 to a concentration means 26.

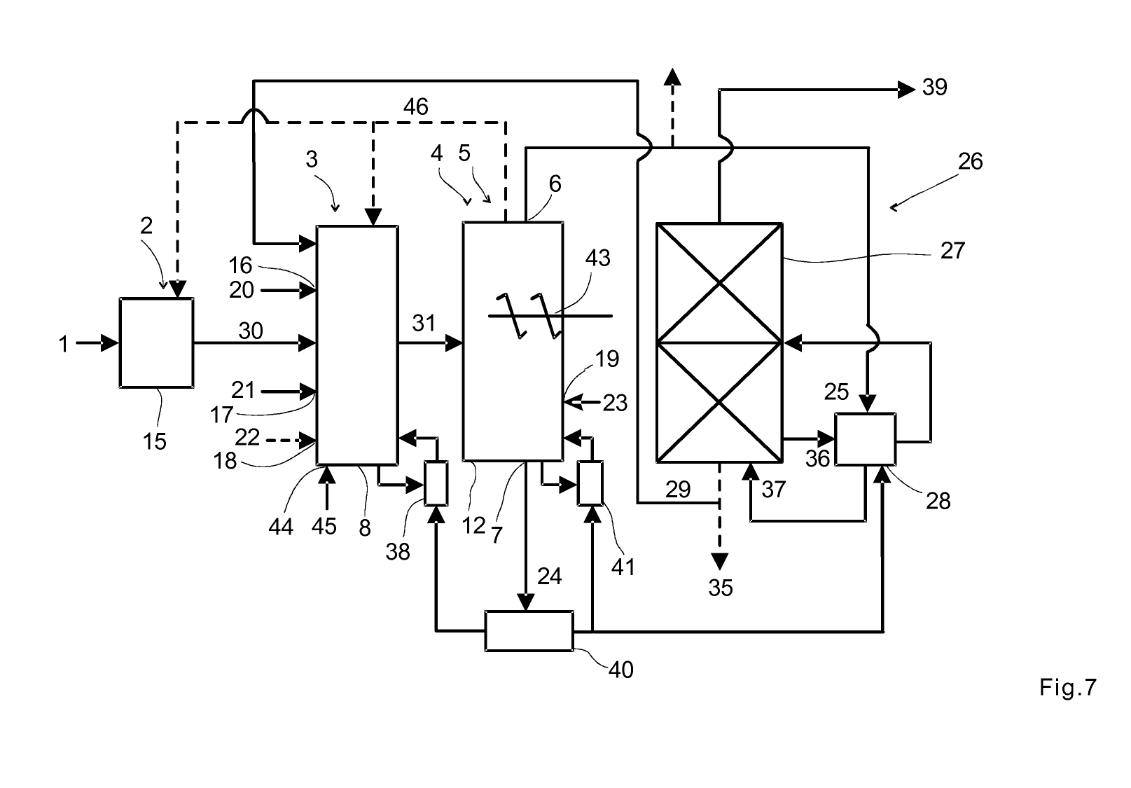

In a seventh preferred embodiment of the apparatus of the invention shown in FIG. 7 said crushing means 2 comprises a separate crusher 15 for crushing waste 1 to form fine-divided waste 30 of the waste 1.

In this seventh preferred embodiment of the invention said hydrolyzing means 3 comprises a separate hydrolyzing vessel 8 for saccharifying with saccharifying enzymes said fine-divided waste 30 to form saccharified fine-divided waste 31.

In this seventh preferred embodiment of the invention said fermentation means 4 and said vaporization and dehydrating means 5 comprise a combined fermentation and vaporization and dehydration vessel 12 for firstly fermenting with ethanol-producing microorganisms 23 said saccharified fine-divided waste 31 to form fermented fine-divided waste 32 containing mixture of ethanol and water 25 and for thereafter at least partly separation by vaporization said mixture of ethanol and water 25 from said fermented fine-divided waste 32 and for dehydrating said fermented fine-divided waste 32 to form dry matter 24 of said fermented fine-divided waste 32.

The apparatus shown in FIG. 7 comprises conduit means (not marked with a reference numeral) for feeding fine-divided waste 30 from said separate crusher 15 to said separate hydrolyzing vessel 8.

The apparatus shown in FIG. 7 comprises in addition conduit means (not marked with a reference numeral) for feeding saccharified fine-divided waste 31 from said separate hydrolyzing vessel 8 to said combined fermentation and vaporization and dehydration vessel 12.

The apparatus shown in FIG. 7 comprises in addition conduit means (not marked with a reference numeral) for feeding partly dehydrated fermented fine-divided waste 46 from said combined fermentation and vaporization and dehydration vessel 12 to said separate crusher 15 and to said separate hydrolyzing vessel 8. The apparatus shown in FIG. 2 does more preferably not comprise any conduit means for feeding partly dehydrated fermented fine-divided waste 46 from said combined fermentation and vaporization and dehydration vessel 12 to said separate hydrolyzing vessel 8 or to said separate crusher 15.

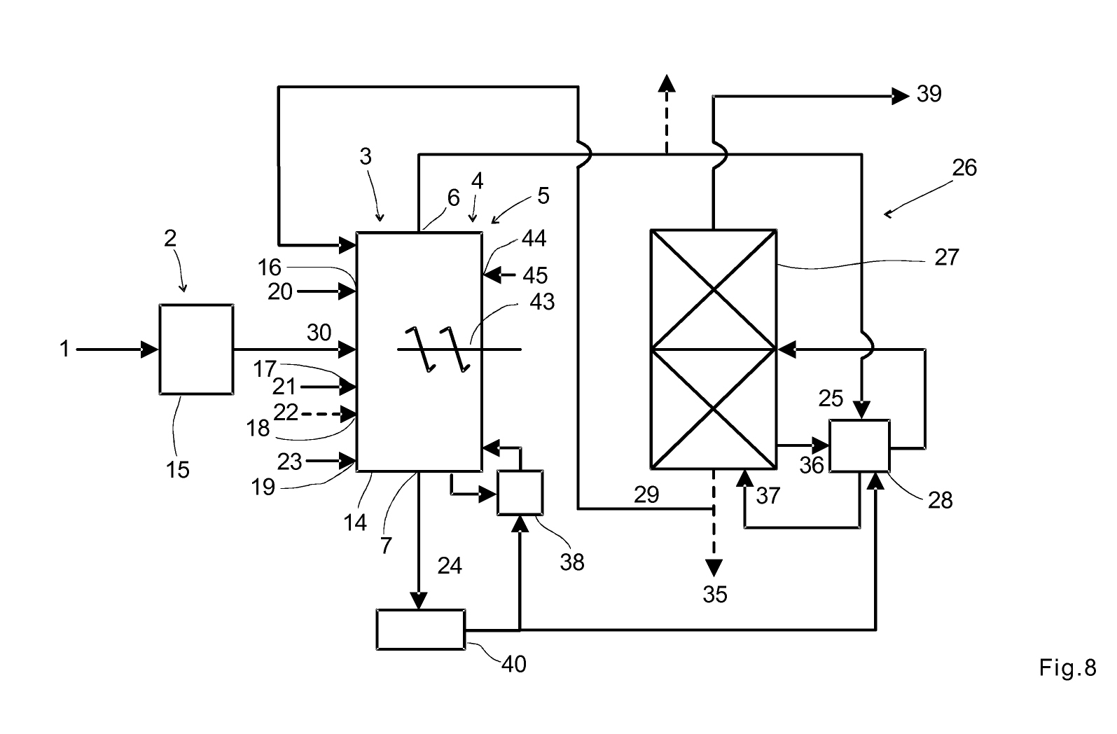

In an eight preferred embodiment of the apparatus of the invention shown in FIG. 8 said crushing means 2 comprises a separate crusher 15 for crushing waste 1 to form fine-divided waste 30 of the waste 1.

Said hydrolyzing means 3 and said fermentation means 4 and said vaporization and dehydrating means 5 comprise in this eight preferred embodiment of the invention shown in FIG. 4 a combined hydrolyzing and fermentation and vaporization and dehydration vessel 14 for firstly saccharifying with saccharifying enzymes said fine-divided waste 30 to form saccharified fine-divided waste 31 and thereafter for fermenting with an ethanol-producing micro-organism 23 such as yeast or a fermenting microorganism said saccharified fine-divided waste 31 to form fermented fine-divided waste 32 containing mixture of ethanol and water 25 and thereafter for at least partly separating by vaporization of said mixture of ethanol and water 25 from said fermented fine-divided waste 32 and for dehydrating said fermented fine-divided waste 32 to form dry matter 24 of said fermented fine-divided waste 32.

The apparatus shown in FIG. 8 comprises conduit means (not marked with a reference numeral) for feeding fine-divided waste 30 from said separate crusher 15 to said combined hydrolyzing and fermentation and vaporization and dehydration vessel 14.

Said crushing means 2 is preferably configured to finely divide said municipal biodegradable waste 1 comprising at least one of starch and cellulose to fine-divided waste 30 having a particle size of 0.5 to 2 mm, for example about 1 mm.

Said hydrolyzing means 3 includes preferably, but not necessarily, means for adding saccharifying enzymes 16 to said fine-divided waste 30, the saccharifying enzymes 20 being selected from amylases, cellulases, and hemicellulases. Amylases are hydrolytic enzymes, which hydrolyze starch and include α-amylases, β-amylases and glycoamylases. Cellulases hydrolyzing cellulose include endoglucanases, cellobiohydrolases and β-glucosidases. Enzymes degrading hemicellulose include xylanases and mannanases and side-chain cleaving enzymes, such as α-glucuronidases, acetyl xylan esterases, α-arabinofuranosidases and α-galactosidases. After combined action of these enzymes, the starch, cellulose and hemicellulose polymers are hydrolyzed into monomeric hexose and pentose sugars, which then may be fermented by ethanol-producing microorganisms. Different enzyme mixtures for hydrolyzing starch, cellulose and hemicellulose polymers are commercially available.

Said means for adding saccharifying enzymes 16 to said biodegradable waste comprising at least one of starch and cellulose can be arranged in combination with a separate hydrolyzing vessel 8 as shown in FIG. 1, 2, 5, or 7, or in combination with a combined hydrolyzing and fermentation vessel 13 as shown in FIGS. 3 and 6, or in combination with a combined hydrolyzing and fermentation and vaporization and dehydration vessel 14 as shown in FIGS. 4 and 8.

Said hydrolyzing means 3 includes preferably, but not necessarily, means for adding other hydrolyzing enzymes 44 to said fine-divided waste 30, such hydrolyzing enzymes 45 being selected from proteases and pectinases.

Said hydrolyzing means 3 includes preferably, but not necessarily, means for adding water 18 to said biodegradable waste comprising at least one of starch and cellulose.

Said means for adding water to said biodegradable waste comprising at least one of starch and cellulose can be arranged in combination with a separate hydrolyzing vessel 8 as shown in FIG. 1, 2, 5, or 7 or in combination with a combined hydrolyzing and fermentation vessel 13 as shown in FIGS. 3 and 6, or in combination with a combined hydrolyzing and fermentation and vaporization and dehydration vessel 14 as shown in FIGS. 4 and 8.

Said hydrolyzing means 3 includes preferably, but not necessarily, means for adding acid 17 to said fine-divided waste 30, the acid 21 being selected from organic acid and mineral acid, such as sulfuric acid.

Said means for adding acid 17 to said biodegradable waste comprising at least one of starch and cellulose can be arranged in combination with a separate hydrolyzing vessel 8 as shown in FIG. 1, 2, 5, or 7, or in combination with a combined hydrolyzing and fermentation vessel 13 as shown in FIGS. 3 and 6, or in combination with a combined hydrolyzing and fermentation and vaporization and dehydration vessel 14 as shown in FIGS. 4 and 8.

Said fermentation means 4 includes preferably, but not necessarily, means for adding an ethanol producing or fermenting microorganism to said saccharified fine-divided waste 31.

Said means for adding an ethanol producing or fermenting microorganism 19 to said saccharified fine-divided waste 31 can be arranged in combination with a separate fermentation vessel 10 as shown in FIGS. 1 and 5, or in combination with a combined hydrolyzing and fermentation vessel 13 as shown in FIGS. 3 and 6, or in combination with a combined fermentation and vaporization and dehydration vessel 12 as shown in FIGS. 2 and 7, or in combination with a combined hydrolyzing and fermentation and vaporization and dehydration vessel 14 as shown in FIGS. 4 and 8. Such ethanol-producing micro-organisms 23 include the baker's yeast Saccharomyces cerevisiae. Due to the complex nature of the carbohydrates present in the municipal biodegradable waste, recombinant yeasts and bacteria may be used, which ferment also pentose sugars and yield ethanol from a wide spectrum of starting material. Such genetically engineered ethanol-producing microorganisms include Saccharomyces cerevisiae, Zymomonas mobilis and Escherichia coli. Preferably, the ethanol-producing microorganisms 23 are added to said saccharified fine-divided waste 31 together with appropriate nutrients, minerals and salts. Preferably, said microorganisms are not in immobilized form and they are not recycled.

In the preferred embodiments of the invention shown in the figures, the apparatus comprises a concentration means 26 for concentrating said mixture of ethanol and water 25.

Said concentration means 26 comprises in the figures an evaporator 27, which preferably is a falling film evaporator or a flash evaporator. A boiler 28 is functionally connected to the evaporator 27. The boiler 28 is adapted to receive a mixture of ethanol and water 25 from the vaporization and dehydrating means 5 and to evaporate a mixture of ethanol and water 25 prior to feeding a mixture of ethanol and water 25 into the evaporator. The apparatus comprises also conduit means (not marked with a reference numeral) for leading condensed evaporate 37 from the evaporator 27 to the boiler 28 to evaporate the condensed evaporate and conduit means (not marked with a reference numeral) for leading heated evaporate 36 from the boiler 28 back to the evaporator 27.

The apparatus shown in the figures comprises conduit means (not marked with a reference numeral) for feeding a residual bottom stream of distilled water 29 from the evaporator 27 to the hydrolyzing means 2. Alternatively or in addition distilled water 35 can be removed from the process as shown with a dotted arrow in the figures.

The apparatus shown in the figures comprises conduit means (not marked with a reference numeral) for feeding concentrated mixture of ethanol and water 39 from the evaporator 27 for example to a storage tank (not shown in the figures).

In the preferred embodiments of the invention shown in the figures the apparatus comprises a burning means 40 for receiving dry matter 24 and for producing of thermal energy by burning said dry matter 24, and in that the thermal energy is at least partly used in the apparatus.

In the first and fifth embodiments of the invention shown in FIGS. 1 and 5 the apparatus comprises a first heater 38 configured for receiving thermal energy from said burning means 40 and configured for heating the separate hydrolyzing vessel 8. In the first and fifth embodiments of the invention shown in FIGS. 1 and 5 the apparatus comprises a second heater 41 configured for receiving thermal energy from said burning means 40 and configured for heating the separate fermentation vessel 10. In the first and fifth embodiments of the invention shown in FIGS. 1 and 5 the apparatus comprises a third heater 42 configured for receiving thermal energy from said burning means 40 and configured for heating the separate vaporization and dehydration vessel 11. Said first heater 38, said second heater 41 and said third heater 42 comprises preferably a heat exchanger (not shown in the figures).

In the second and sixth embodiments of the invention shown in FIGS. 2 and 6 the apparatus comprises a first heater 38 configured for receiving thermal energy from said burning means 40 and configured for heating the separate hydrolyzing vessel 8. In the second and sixth embodiments of the invention shown in FIGS. 2 and 4 the apparatus comprises a second heater 41 configured for receiving thermal energy from said burning means 40 and configured for heating the combined fermentation and vaporization and dehydration vessel 12. Said first heater 38 and said second heater 41 comprises preferably a heat exchanger (not shown in the figures).

In the third and seventh embodiments of the invention shown in FIGS. 3 and 7 the apparatus comprises a first heater 38 configured for receiving thermal energy from said burning means 40 and configured for heating the combined hydrolyzing and fermentation vessel 8. In the third and seventh embodiments of the invention shown in FIGS. 3 and 7 the apparatus comprises a second heater 41 configured for receiving thermal energy from said burning means 40 and configured for heating the separate vaporization and dehydration vessel 11. Said first heater 38 and said second heater 41 comprises preferably a heat exchanger (not shown in the figures).

In the fourth and eighth embodiments of the invention shown in FIGS. 4 and 8 the apparatus comprises a first heater 38 configured for receiving thermal energy from said burning means 40 and configured for heating the combined hydrolyzing and fermentation and vaporization and dehydration vessel 14. Said first heater 38 comprises preferably a heat exchanger (not shown in the figures).

In the embodiments of the invention shown in the figures thermal energy from said burning means can also be fed to the boiler 28.

The invention also relates to a method for processing waste 1 containing fermentable raw materials selected from sugars and raw materials such as starch and cellulose capable of being saccharified into fermentable sugars.

The waste 1 is preferably, but not necessarily, municipal biodegradable waste containing fermentable raw materials selected from sugars and raw materials such as starch and cellulose capable of being saccharified into fermentable sugars.

The method comprises a crushing step for at least partly fine-dividing said waste 1 containing fermentable raw materials selected from sugars and raw materials such as starch and cellulose capable of being saccharified into fermentable sugars to form fine-divided waste 30.

A sorting step for removing non-fermentable matter such as plastics and bones from the waste 1 can preferably, but not necessarily, be performed before said crushing step.

Said crushing step is preferably, but not necessarily, performed with a crushing means 2 configured to also fine-divide possible non-fermentable matter such as plastics and bones present in the waste 1.

The method can in addition comprise a screening step for screening said fine-divided waste 30 to remove possible non-fermentable matter such as plastics and bones present in the fine-divided waste 30 after said crushing step for at least partly fine-dividing said waste 1.

The method comprises a saccharification step for at least partly saccharifying with enzymes 20 said fine-divided waste 30 to form saccharified fine-divided waste 31. In this saccharification step the saccharifying enzymes break down at least raw materials such as starch and cellulose capable of being saccharified into fermentable sugars present in the fine-divided waste 30 to produce fermentable sugars.

The method comprises a fermentation step for at least partly fermenting with an ethanol-producing microorganism 23 said saccharified fine-divided waste 31 to form fermented fine-divided waste 32 containing a mixture of ethanol and water 25. In this fermentation step the ethanol-producing microorganisms use fermentable sugars in the saccharified fine-divided waste to produce ethanol i.e. to produce said fermented fine-divided waste 32 containing a mixture of ethanol and water 25.

The method comprises a vaporization step for at least partly separate by vaporization said mixture of ethanol and water 25 from said fermented fine-divided waste 32 that can be performed in a vaporization and dehydration means 5. Alternatively, the method comprises a vaporization step for at least partly separate by vaporization said mixture of ethanol and water 25 from said fermented fine-divided waste 32 in a separate vaporization means 51.

The method comprises a dehydrating step for at least partly dehydrating said fermented fine-divided waste 32 to form dry matter 24 of said fermented fine-divided waste 32 that can be performed in a vaporization and dehydration means 5. Alternatively, the method comprises a vaporization step for at least partly dehydrating said fermented fine-divided waste 32 to form dry matter 24 of said fermented fine-divided waste 32 in a separate dehydration means 52.

The method comprises an ethanol mixture collecting step for discharging and collecting said vaporized mixture of ethanol and water 25 from said vaporization and dehydrating means 5 or from said separate vaporization means 51.

The method comprises a dry matter 24 collecting step for collecting said dry matter 24 from said vaporization and dehydrating means 5 or from said separate dehydration means 52.

Said fermented fine-divided waste 32 is preferably, but not necessarily, mixing and/or moving said fermented fine-divided waste 32 during at least one of the vaporization steps and the dehydrating steps. Said mixing or moving can for example be performed by means of a rotary dryer.

The fermentation step of the method of the invention may comprise a pre-vaporization step for removing fermentation inhibitors from fermented fine-divided waste containing fermentation inhibitors which includes feeding step for firstly feeding fermented fine-divided waste containing fermentation inhibitors from a fermentation vessel to a vaporization and dehydration vessel to perform to a pre-evaporation step to evaporate fermentation inhibitors such as excess volatile inhibiting compounds, such as ethanol, lactic acid and acetic acid, which inhibits fermentation in the fermentation vessel off from fermented fine-divided waste with the vaporization and dehydrating vessel, and comprise a feeding step for after said pre-evaporation step feeding back partly evaporated fermented fine-divided waste that has a reduced amount of fermentation inhibitors from the vaporization and dehydration vessel to the fermentation vessel for further or additional fermentation of the partly evaporated fermented fine-divided waste, resulting in higher yield and fermentation rate. In other words, the fermentation step of the method of the invention may for example comprise a pre-vaporization step for removing fermentation inhibitors from fermented fine-divided waste containing fermentation inhibitors by vaporization fermentation inhibitors from said fermented fine-divided waste containing fermentation inhibitors.

The saccharification step can take 1-24 hours depending for example on the amount of fine-divided waste

The fermentation step can take 24-72 hours and be performed at a temperature between about 20° C. and about 40° C. depending for example on the ethanol-producing microorganism.

The method of the invention may comprise a solid matter separation step that is performed after the fermentation step and before the vaporization step and that us performed in a solid matter separator means 53. FIGS. 13 to 16 show the principle of such methods. In the solid matter separation step fermented fine divided waste 32 is for example mechanically pressed to separate fermentation beer 55 from the fermented fine divided waste 32. In the solid matter separation step the other product obtained that is fermented fine divided waste 32, from which fermentation beer 55 has been separated, can be considered to partly dehydrated fermented fine-divided waste 46, as illustrated in FIGS. 13 to 16. If the method of the invention comprises a such solid matter separation step, the fermentation beer 55 obtained is preferably subjected to a evaporation step in a evaporation means 54, in which evaporation step mixture of ethanol and water 25 is separated from the fermentation beer 55. If the method comprises a such evaporation step, a residual bottom stream 56, that is fermentation beer 55 from which mixture of ethanol and water 25 has been removed, is preferably returned into the process for example by feeding and using the residual bottom stream in at least one of the following steps: the saccaharification step and the fermentation step. If the method comprises a such solid matter separation step, partly dehydrated fermented fine-divided waste 46 obtained from the solid matter separation step is further processed by performing said dehydrating step to the partly dehydrated fermented fine-divided waste 46. If the method comprises a such solid matter separation step, partly dehydrated fermented fine-divided waste 46 obtained from the solid matter separation step can be used in the fermentation step. The evaporator step is preferably performed by using a falling film evaporator that is configured to divide fermentation beer 55 into mixture of ethanol and water 25 and into a residual bottom stream 56. If the fermentation beer obtaining from the solid matter separation step is subjected to a evaporating step, the residual bottom stream 56 is preferably used in at least one of the following: the crushing step, the saccaharification step or the fermentation step.

In a first preferred embodiment of the method of the invention shown in FIG. 1, the method includes a feeding step for feeding waste 1 into a separate hydrolyzing vessel 8.

In this first preferred embodiment of the invention said crushing step includes crushing said waste 1 with a crusher 9 to form said fine-divided waste 30 with a crusher 9, which is arranged in said separate hydrolyzing vessel 8.

This first preferred embodiment of the invention includes a saccharification step performed in the separate hydrolyzing vessel 8 for saccharifying with saccharifying enzymes said fine-divided waste 30 to form saccharified fine-divided waste 31.

This first preferred embodiment of the invention includes a feeding step for feeding saccharified fine-divided waste 31 from said separate hydrolyzing vessel 8 into a separate fermentation vessel 10.

This first preferred embodiment of the invention includes a fermentation step performed in the separate fermentation vessel 10 for at least partly fermenting with an ethanol-producing microorganism said saccharified fine-divided waste 31 to obtain fermented fine-divided waste 32 containing mixture of ethanol and water 25.

This first preferred embodiment of the invention includes a feeding step for feeding fermented fine-divided waste 32 containing a mixture of ethanol and water 25 from said separate fermentation vessel 10 into a separate vaporization and dehydration vessel 11.

This first preferred embodiment of the invention includes a vaporization step and a dehydration step performed in the separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32.

This first preferred embodiment of the invention includes an ethanol mixture collecting step for discharging and collecting said vaporized mixture of ethanol and water 25 from said separate vaporization and dehydration vessel 11.

This first preferred embodiment of the invention includes a dry matter 24 collecting step for discharging and collecting said dry matter 24 from said separate vaporization and dehydration vessel 11.

The fermentation step of this first preferred embodiment of the method of the invention illustrated in FIG. 1 may include performing a pre-vaporization step for removing fermentation inhibitors from fermented fine-divided waste containing fermentation inhibitors 47. This pre-vaporization step includes a feeding step for feeding fermented fine-divided waste containing fermentation inhibitors 47 from said separate fermentation vessel 10 to the separate vaporization and dehydration vessel 11. This pre-vaporization step includes a pre-evaporation step for separating by vaporization in the separate vaporization and dehydration vessel 11 fermentation inhibitors from said fine-divided waste containing fermentation inhibitors 47 whereby partly dehydrated fermented fine-divided waste 46 is obtained. This pre-vaporization step includes a feeding step for feeding partly dehydrated fermented fine-divided waste 46 from said separate vaporization and dehydration vessel 11 into at least one of the separate hydrolyzing vessel 8 and the separate fermentation vessel 10. This pre-vaporization step includes preferably a feeding step for feeding partly dehydrated fermented fine-divided waste 46 from said separate vaporization and dehydration vessel 11 only to said separate fermentation vessel 10.

FIG. 9 shows a ninth preferred embodiment of the method of the invention that otherwise corresponds to the first preferred embodiment of the method of the invention shown in FIG. 1 with the exception that instead of a separate vaporization and dehydration vessel 11, a separate vaporization vessel 48 and a separate dehydration vessel 49 is used. This ninth preferred embodiment of the invention includes a feeding step for feeding fermented fine-divided waste 32 containing a mixture of ethanol and water 25 from said separate fermentation vessel 10 into said separate vaporization vessel 48. This ninth preferred embodiment of the invention includes a vaporization step performed in the separate vaporization vessel 48 for at least partly separating by vaporization said mixture of ethanol and water 25 from said fermented fine-divided waste 32 and to form ethanol-free fermented fine-divided waste 50 in said separate vaporization vessel 49. The ninth preferred embodiment of the method of the invention comprises a step for feeding ethanol-free fermented fine-divided waste 50 from the separate vaporization vessel 48 to the separate dehydration vessel 49. This ninth preferred embodiment of the method of the invention includes a dehydration step performed in the separate dehydration vessel 49 dehydrating said ethanol-free fermented fine-divided waste 50 to form dry matter 24 of said ethanol-free fermented fine-divided waste 50. This ninth preferred embodiment of the invention includes an ethanol mixture collecting step for discharging and collecting said vaporized mixture of ethanol and water 25 from said separate vaporization vessel 48. This ninth preferred embodiment of the invention includes a dry matter 24 collecting step for discharging and collecting said dry matter 24 from said separate dehydration vessel 49.

In the same manner as the first preferred embodiment of the method of the invention the ninth preferred embodiment of the method of the invention may comprises a pre-evaporation step for removing fermentation inhibitors from fermented fine-divided waste containing fermentation inhibitors 47. In the ninth preferred embodiment of the method of the invention fermentation inhibitors are removed by feeding fermented fine-divided waste containing fermentation inhibitors 47 into the separate vaporization vessel 48 and by using the separate vaporization vessel 48 for vaporizing fermentation inhibitors off the fermented fine-divided waste containing fermentation inhibitors 47.

The method may as in the thirteenth embodiment shown in FIG. 13 that otherwise essentially corresponds to the first embodiment shown in FIG. 1, comprise a solid matter separation step that is performed in solid matter separator means 53 after the fermentation step that is performed in the separate fermentation vessel 10 and before the vaporization step that is performed in the separate vaporization and dehydration vessel 11. In this thirteenth embodiment of the method of the invention fermented fine divided waste 32 is in the solid matter separator means 53 separated into fermentation beer 55 or ethanol beer that is fed into a evaporator means 54 and into partly dehydrated fermented fine-divided waste 46 that is fed into at least one of the separate vaporization and dehydration vessel 11 to be used in the vaporization step and the dehydrations step and the separate fermentation vessel 10 to be used in the fermentation step. In the evaporator means 54 the fermentation beer 55 or ethanol beer is subjected to a evaporation step for dividing fermentation beer 55 into mixture of ethanol and water 25 that in FIG. 13 is fed to concentration means 26 for performing a concentration step and into a residual bottom stream 56 that is fed into at least one of the separate hydrolyzing vessel 8 to be used in the saccarification step and the separate fermentation vessel 10 to be used in the fermentation step.

In a second preferred embodiment of the method of the invention shown in FIG. 2, the method includes a feeding step for feeding waste 1 into a separate hydrolyzing vessel 8.

In this second preferred embodiment of the invention said crushing step includes crushing said waste 1 with a crusher 9 to form fine-divided waste 30 with a crusher 9, which is arranged in said separate hydrolyzing vessel 8.

This second preferred embodiment of the invention includes a saccharification step performed in the separate hydrolyzing vessel 8 for saccharifying with saccharifying enzymes said fine-divided waste 30 to form saccharified fine-divided waste 31.

This first preferred embodiment of the invention includes a feeding step for feeding saccharified fine-divided waste 31 from said separate hydrolyzing vessel 8 into a combined fermentation and vaporization and dehydration vessel 12.

In this second preferred embodiment of the invention shown in FIG. 2, the method includes firstly a fermentation step performed in the combined fermentation and vaporization and dehydration vessel 12 for at least partly fermenting with an ethanol-producing microorganism said saccharified fine-divided waste 31 to obtain fermented fine-divided waste 32 containing mixture of ethanol and water 25 in the combined fermentation and vaporization and dehydration vessel 12.

In the second preferred embodiment of the invention shown in FIG. 2, the method includes thereafter a vaporization and a dehydration step performed in the combined fermentation and vaporization and dehydration vessel 12 for at least partly separating by vaporization said mixture of ethanol and water 25 from said fermented fine-divided waste 32 and for dehydrating said fermented fine-divided waste 32 to form dry matter 24 of said fermented fine-divided waste 32 in the combined fermentation and vaporization and dehydration vessel 12.

This second preferred embodiment of the invention includes an ethanol mixture collecting step for discharging and collecting said vaporized mixture of ethanol and water 25 from said combined fermentation and vaporization and dehydration vessel 12.

This second preferred embodiment of the invention includes a dry matter 24 collecting step for discharging and collecting said dry matter 24 from said combined fermentation and vaporization and dehydration vessel 12.

The fermentation step of this second preferred embodiment of the method of the invention illustrated in FIG. 2 may include performing a pre-vaporization step for removing fermentation inhibitors from fermented fine-divided waste containing fermentation inhibitors 47. This pre-vaporization step includes a pre-evaporation step for separating in said combined fermentation and vaporization and dehydration vessel 12 by vaporization fermentation inhibitors from said fine-divided waste containing fermentation inhibitors 47 whereby partly dehydrated fermented fine-divided waste 46 is obtained. The pre-vaporization step may include performing a feeding step for feeding partly dehydrated fermented fine-divided waste 46 from said combined fermentation and vaporization and dehydration vessel 12 into said separate hydrolyzing vessel 8. The pre-vaporization step may include continuing said fermentation step in said combined fermentation and vaporization and dehydration vessel 12 after performing said pre-vaporization step by at least partly fermenting with ethanol-producing microorganism 23 said partly dehydrated fermented fine-divided waste 46 in said combined fermentation and vaporization and dehydration vessel 12.

In a third preferred embodiment of the method of the invention shown in FIG. 3, the method includes a feeding step for feeding waste 1 into a combined hydrolyzing and fermentation vessel 13.

In this third preferred embodiment of the invention said crushing step includes crushing said waste 1 with a crusher 9 to form fine-divided waste 30 with a crusher 9, which is arranged in said combined hydrolyzing and fermentation vessel 13.

This third preferred embodiment of the method of the invention includes firstly a saccharification step performed in said combined hydrolyzing and fermentation vessel 13 for saccharifying with saccharifying enzymes said fine-divided waste 30 to form saccharified fine-divided waste 31. This third preferred embodiment of the invention includes thereafter a fermentation step in the combined hydrolyzing and fermentation vessel 13 for at least partly fermenting with an ethanol-producing microorganism said saccharified fine-divided waste 31 to obtain fermented fine-divided waste 32 containing mixture of ethanol and water 25 in the combined hydrolyzing and fermentation vessel 13.

This third preferred embodiment of the method of the invention includes a feeding step for feeding fermented fine-divided waste 32 containing mixture of ethanol and water 25 from said combined hydrolyzing and fermentation vessel 13 into a separate vaporization and dehydration vessel 11.

In the third preferred embodiment of the method of the invention shown in FIG. 3, the method includes a dehydration step performed in the separate vaporization and dehydration vessel 11 for at least partly separating by vaporization said mixture of ethanol and water 25 from said fermented fine-divided waste 32 and for dehydrating said fermented fine-divided waste 32 to form dry matter 24 of said fermented fine-divided waste 32 in the separate vaporization and dehydration vessel 11.

This third preferred embodiment of the method of the invention includes an ethanol mixture collecting step for discharging and collecting said vaporized mixture of ethanol and water 25 from said separate vaporization and dehydration vessel 11.

This third preferred embodiment of the method of the invention includes a dry matter 24 collecting step for discharging and collecting said dry matter 24 from said separate vaporization and dehydration vessel 11.

The fermentation step of this third preferred embodiment of the method of the invention may as illustrated in FIG. 3 include performing a pre-vaporization step for removing fermentation inhibitors from fermented fine-divided waste containing fermentation inhibitors 47. This pre-vaporization step includes a feeding step for feeding fermented fine-divided waste containing fermentation inhibitors 47 from said combined hydrolyzing and fermentation vessel 13 to the separate vaporization and dehydration vessel 11. This pre-vaporization step includes a pre-evaporation step for separating by vaporization in the separate vaporization and dehydration vessel 11 fermentation inhibitors from fine-divided waste containing fermentation inhibitors 47 whereby partly dehydrated fermented fine-divided waste 46 is obtained. This pre-vaporization step includes a feeding step for feeding partly dehydrated fermented fine-divided waste 46 from said separate vaporization and dehydration vessel 11 into said combined hydrolyzing and fermentation vessel 13.

FIG. 10 shows a tenth preferred embodiment of the method of the invention that otherwise corresponds to the third preferred embodiment of the method of the invention shown in FIG. 3 with the exception that instead of a separate vaporization and dehydration vessel 11, a separate vaporization vessel 48 and a separate dehydration vessel 49 is used. This tenth preferred embodiment of the invention includes a feeding step for feeding fermented fine-divided waste 32 containing a mixture of ethanol and water 25 from said separate fermentation vessel 10 into said separate vaporization vessel 48. This tenth preferred embodiment of the invention includes a vaporization step performed in the separate vaporization vessel 48 for at least partly separating by vaporization said mixture of ethanol and water 25 from said fermented fine-divided waste 32 and to form ethanol-free fermented fine-divided waste 50 in said separate vaporization vessel 49. The tenth preferred embodiment of the method of the invention comprises a step for feeding ethanol-free fermented fine-divided waste 50 from the separate vaporization vessel 48 to the separate dehydration vessel 49. This tenth preferred embodiment of the method of the invention includes a dehydration step performed in the separate dehydration vessel 49 dehydrating said ethanol-free fermented fine-divided waste 50 to form dry matter 24 of said ethanol-free fermented fine-divided waste 50. This tenth preferred embodiment of the invention includes an ethanol mixture collecting step for discharging and collecting said vaporized mixture of ethanol and water 25 from said separate vaporization vessel 48. This tenth preferred embodiment of the invention includes a dry matter 24 collecting step for discharging and collecting said dry matter 24 from said separate dehydration vessel 49.

In the same manner as the third preferred embodiment of the method of the invention the tenth preferred embodiment of the method of the invention may comprises a pre-evaporation step for removing fermentation inhibitors from fermented fine-divided waste containing fermentation inhibitors 47. In the tenth preferred embodiment of the method of the invention fermentation inhibitors are removed by feeding fermented fine-divided waste containing fermentation inhibitors 47 into the separate vaporization vessel 48 and by using the separate vaporization vessel 48 for vaporizing fermentation inhibitors off the fermented fine-divided waste containing fermentation inhibitors 47.

The method may as in the fourteenth embodiment shown in FIG. 14 that otherwise essentially corresponds to the third embodiment shown in FIG. 3, comprise a solid matter separation step that is performed in solid matter separator means 53 after the saccharification step and fermentation step that is performed in the combined hydrolyzing and fermentation vessel 13 and before the vaporization step that is performed in the separate vaporization and dehydration vessel 11. In this fourteenth embodiment of the method of the invention fermented fine divided waste 32 is in the solid matter separator means 53 separated into fermentation beer 55 or ethanol beer that is fed into a evaporator means 54 and into partly dehydrated fermented fine-divided waste 46 that is fed into at least one of the separate vaporization and dehydration vessel 11 to be used in the vaporization step and the dehydrations step and the combined hydrolyzing and fermentation vessel 13 to be used in the saccaharification step and fermentation step. In the evaporator means 54 the fermentation beer 55 or ethanol beer is subjected to a evaporation step for dividing fermentation beer 55 into mixture of ethanol and water 25 that in FIG. 14 is fed to concentration means 26 for performing a concentration step and into a residual bottom stream 56 that is fed into at least one of the separate hydrolyzing vessel 8 to be used in the saccarification step and the combined hydrolyzing and fermentation vessel 13 to be used in the saccharification step and/or the fermentation step.

In a fourth preferred embodiment of the method of the invention the method shown in FIG. 4, the method includes a feeding step for feeding said waste 1 into a combined hydrolyzing and fermentation and vaporization and dehydration vessel 14.

In this fourth preferred embodiment of the method of the invention said crushing step includes crushing said waste 1 with a crusher 9 to form fine-divided waste 30 with a crusher 9, which is arranged in said combined hydrolyzing and fermentation and vaporization and dehydration vessel 14.

This fourth preferred embodiment of the method of the invention includes firstly a saccharification step performed in said combined hydrolyzing and fermentation and vaporization and dehydration vessel 14 for saccharifying with saccharifying enzymes said fine-divided waste 30 to form saccharified fine-divided waste 31.

This fourth preferred embodiment of the method of the invention includes thereafter a fermentation step performed in said combined hydrolyzing and fermentation and vaporization and dehydration vessel 14 for at least partly fermenting with an ethanol-producing microorganism said saccharified fine-divided waste 31 to obtain fermented fine-divided waste 32 containing mixture of ethanol and water 25 in the combined hydrolyzing and fermentation and vaporization and dehydration vessel 14.

This fourth preferred embodiment of the method of the invention includes thereafter a dehydration step performed in said combined hydrolyzing and fermentation and vaporization and dehydration vessel 14 for at least partly separating by vaporization said mixture of ethanol and water 25 from said fermented fine-divided waste 32 and for dehydrating said fermented fine-divided waste 32 to form dry matter 24 of said fermented fine-divided waste 32 in said combined hydrolyzing and fermentation and vaporization and dehydration vessel 14.

This fourth preferred embodiment of the method of the invention includes an ethanol mixture collecting step for discharging and collecting said vaporized mixture of ethanol and water 25 from said combined hydrolyzing and fermentation and vaporization and dehydration vessel 14.

This fourth preferred embodiment of the method of the invention includes a dry matter 24 collecting step for discharging and collecting said dry matter 24 from said combined hydrolyzing and fermentation and vaporization and dehydration vessel 14.

The fermentation step of this fourth preferred embodiment of the method of the invention may include a pre-vaporization step for removing fermentation inhibitors from fermented fine-divided waste containing fermentation inhibitors 47. This pre-vaporization step includes pre-vaporization step includes a pre-evaporation step for separating in said combined hydrolyzing and fermentation and vaporization and dehydration vessel 14 by vaporization fermentation inhibitors from said fine-divided waste containing fermentation inhibitors 47 whereby partly dehydrated fermented fine-divided waste 46 is obtained. This pre-vaporization step includes by continuing said fermentation step in said combined hydrolyzing and fermentation and vaporization and dehydration vessel 14 after said pre-vaporization step by at least partly fermenting with ethanol-producing microorganism 23 said partly dehydrated fermented fine-divided waste 46 in said combined hydrolyzing and fermentation and vaporization and dehydration vessel 14.

In a fifth preferred embodiment of the method of the invention shown in FIG. 5 the method includes a feeding step for feeding municipal biodegradable waste 1 into a separate crusher 15.

This fifth preferred embodiment of the method of the invention includes a crushing step including crushing municipal biodegradable waste 1 with said separate crusher 15 to form fine-divided waste 30.

This fifth preferred embodiment of the invention includes a feeding step for feeding said fine-divided waste 30 from said separate crusher 15 into a separate hydrolyzing vessel 8.

This fifth preferred embodiment of the method of the invention includes a saccharification step performed in said separate hydrolyzing vessel 8 for at least partly saccharifying with saccharifying enzymes said fine-divided waste 30 to form saccharified fine-divided waste 31.

This fifth preferred embodiment of the invention includes a feeding step for feeding said saccharified fine-divided waste 31 from said separate hydrolyzing vessel 8 into a separate fermentation vessel 10.

This fifth preferred embodiment of the invention includes a fermentation step performed in said separate fermentation vessel 10 for at least partly fermenting with an ethanol-producing microorganism said saccharified fine-divided waste 31 to obtain fermented fine-divided waste 32 containing mixture of ethanol and water 25 in said separate fermentation vessel 10.

This fifth preferred embodiment of the invention includes a feeding step for feeding said fermented fine-divided waste 32 containing a mixture of ethanol and water 25 from said separate fermentation vessel 10 into a separate vaporization and dehydration vessel 11.

This fifth preferred embodiment of the invention includes a vaporization and dehydrating step performed in said separate vaporization and dehydration vessel 11 for at least partly separating by vaporization mixture of ethanol and water 25 from said fermented fine-divided waste 32 and for dehydrating said fermented fine-divided waste 32 to form dry matter 24 of said fermented fine-divided waste 32.

This fifth preferred embodiment of the method of the invention includes an ethanol mixture collecting step for discharging and collecting said vaporized mixture of ethanol and water 25 from said separate vaporization and dehydration vessel 11.

This fifth preferred embodiment of the method of the invention includes a dry matter 24 collecting step for discharging and collecting said dry matter 24 from said separate vaporization and dehydration vessel 11.

The fermentation step of this fifth preferred embodiment of the method of the invention illustrated in FIG. 5 may include performing a pre-vaporization step for removing fermentation inhibitors from fermented fine-divided waste containing fermentation inhibitors 47. This pre-vaporization step includes a feeding step for feeding fermented fine-divided waste containing fermentation inhibitors 47 from said separate fermentation vessel 10 to the separate vaporization and dehydration vessel 11. This pre-vaporization step includes a pre-evaporation step for separating by vaporization in the separate vaporization and dehydration vessel 11 fermentation inhibitors from said fine-divided waste containing fermentation inhibitors 47 whereby partly dehydrated fermented fine-divided waste 46 is obtained. This pre-vaporization step includes a feeding step for feeding partly dehydrated fermented fine-divided waste 46 from said separate vaporization and dehydration vessel 11 into at least one of the separate hydrolyzing vessel 8, the separate fermentation vessel 10 and the separate crusher 15. This pre-vaporization step includes preferably a feeding step for feeding partly dehydrated fermented fine-divided waste 46 from said separate vaporization and dehydration vessel 11 only to said separate fermentation vessel 10.

FIG. 11 shows a eleventh preferred embodiment of the method of the invention that otherwise corresponds to the first preferred embodiment of the method of the invention shown in FIG. 1 with the exception that instead of a separate vaporization and dehydration vessel 11, a separate vaporization vessel 48 and a separate dehydration vessel 49 is used. This eleventh preferred embodiment of the invention includes a feeding step for feeding fermented fine-divided waste 32 containing a mixture of ethanol and water 25 from said separate fermentation vessel 10 into said separate vaporization vessel 48. This eleventh preferred embodiment of the invention includes a vaporization step performed in the separate vaporization vessel 48 for at least partly separating by vaporization said mixture of ethanol and water 25 from said fermented fine-divided waste 32 and to form ethanol-free fermented fine-divided waste 50 in said separate vaporization vessel 49. The eleventh preferred embodiment of the method of the invention comprises a step for feeding ethanol-free fermented fine-divided waste 50 from the separate vaporization vessel 48 to the separate dehydration vessel 49. This eleventh preferred embodiment of the method of the invention includes a dehydration step performed in the separate dehydration vessel 49 dehydrating said ethanol-free fermented fine-divided waste 50 to form dry matter 24 of said ethanol-free fermented fine-divided waste 50. This eleventh preferred embodiment of the invention includes an ethanol mixture collecting step for discharging and collecting said vaporized mixture of ethanol and water 25 from said separate vaporization vessel 48. This eleventh preferred embodiment of the invention includes a dry matter 24 collecting step for discharging and collecting said dry matter 24 from said separate dehydration vessel 49.

In the same manner as the first preferred embodiment of the method of the invention the eleventh preferred embodiment of the method of the invention may comprises a pre-evaporation step for removing fermentation inhibitors from fermented fine-divided waste containing fermentation inhibitors 47. In the eleventh preferred embodiment of the method of the invention fermentation inhibitors are removed by feeding fermented fine-divided waste containing fermentation inhibitors 47 into the separate vaporization vessel 48 and by using the separate vaporization vessel 48 for vaporizing fermentation inhibitors off the fermented fine-divided waste containing fermentation inhibitors 47.

The method may as in the fifteenth embodiment shown in FIG. 15 that otherwise essentially corresponds to the fifth embodiment shown in FIG. 5, comprise a solid matter separation step that is performed in solid matter separator means 53 after the fermentation step that is performed in the separate fermentation vessel 10 and before the vaporization step that is performed in the separate vaporization and dehydration vessel 11. In this fifteenth embodiment of the method of the invention fermented fine divided waste 32 is in the solid matter separator means 53 separated into fermentation beer 55 or ethanol beer that is fed into a evaporator means 54 and into partly dehydrated fermented fine-divided waste 46 that is fed into at least one of the separate vaporization and dehydration vessel 11 to be used in the vaporization step and the dehydrations step and the separate fermentation vessel 10 to be used in the fermentation step. In the evaporator means 54 the fermentation beer 55 or ethanol beer is subjected to a evaporation step for dividing fermentation beer 55 into mixture of ethanol and water 25 that in FIG. 15 is fed to concentration means 26 for performing a concentration step and into a residual bottom stream 56 that is fed into at least one of the separate hydrolyzing vessel 8 to be used in the saccarification step and the separate fermentation vessel 10 to be used in the fermentation step.

In a sixth preferred embodiment of the method of the invention shown in FIG. 6 the method includes a feeding step for feeding municipal biodegradable waste 1 into a separate crusher 15.

This sixth preferred embodiment of the method of the invention includes a crushing step including crushing municipal biodegradable waste 1 with said separate crusher 15 to form fine-divided waste 30.

This sixth preferred embodiment of the method of the invention includes a feeding step for feeding said fine-divided waste 30 from said separate crusher 15 into a combined hydrolyzing and fermentation vessel 13.

This sixth preferred embodiment of the method of the invention includes firstly a saccharification step performed in said combined hydrolyzing and fermentation vessel 13 for at least partly saccharifying with saccharifying enzymes said fine-divided waste 30 to form saccharified fine-divided waste 31 in said combined hydrolyzing and fermentation vessel 13. This sixth preferred embodiment of the method of the invention includes thereafter a fermentation step performed in said combined hydrolyzing and fermentation vessel 13 for at least partly fermenting with ethanol-producing microorganism said saccharified fine-divided waste 31 to obtain fermented fine-divided waste 32 containing mixture of ethanol and water 25 in said combined hydrolyzing and fermentation vessel 13.

This sixth preferred embodiment of the invention includes a feeding step for feeding said fermented fine-divided waste 32 containing a mixture of ethanol and water 25 from said combined hydrolyzing and fermentation vessel 13 into a separate vaporization and dehydration vessel 11.

This sixth preferred embodiment of the invention includes a vaporization and dehydrating step performed in said separate vaporization and dehydration vessel 11 for at least partly separating by vaporization mixture of ethanol and water 25 from said fermented fine-divided waste 32 and for dehydrating said fermented fine-divided waste 32 to form dry matter 24 of said fermented fine-divided waste 32.

This sixth preferred embodiment of the method of the invention includes an ethanol mixture collecting step for discharging and collecting said vaporized mixture of ethanol and water 25 from said separate vaporization and dehydration vessel 11.

This sixth preferred embodiment of the method of the invention includes a dry matter 24 collecting step for discharging and collecting said dry matter 24 from said separate vaporization and dehydration vessel 11.

The fermentation step of this sixth preferred embodiment of the method of the invention may as illustrated in FIG. 6 include performing a pre-vaporization step for removing fermentation inhibitors from fermented fine-divided waste containing fermentation inhibitors 47. This pre-vaporization step includes a feeding step for feeding fermented fine-divided waste containing fermentation inhibitors 47 from said combined hydrolyzing and fermentation vessel 13 to the separate vaporization and dehydration vessel 11. This pre-vaporization step includes a pre-evaporation step for separating by vaporization in the separate vaporization and dehydration vessel 11 fermentation inhibitors from fine-divided waste containing fermentation inhibitors 47 whereby partly dehydrated fermented fine-divided waste 46 is obtained. This pre-vaporization step includes a feeding step for feeding partly dehydrated fermented fine-divided waste 46 from said separate vaporization and dehydration vessel 11 into at least one of said combined hydrolyzing and fermentation vessel 13 and said separate crusher 15. This pre-vaporization step includes preferably a feeding step for feeding partly dehydrated fermented fine-divided waste 46 from said separate vaporization and dehydration vessel 11 only to said combined hydrolyzing and fermentation vessel 13.

FIG. 12 shows a twelfth preferred embodiment of the method of the invention that otherwise corresponds to the first preferred embodiment of the method of the invention shown in FIG. 1 with the exception that instead of a separate vaporization and dehydration vessel 11, a separate vaporization vessel 48 and a separate dehydration vessel 49 is used. This twelfth preferred embodiment of the invention includes a feeding step for feeding fermented fine-divided waste 32 containing a mixture of ethanol and water 25 from said separate fermentation vessel 10 into said separate vaporization vessel 48. This twelfth preferred embodiment of the invention includes a vaporization step performed in the separate vaporization vessel 48 for at least partly separating by vaporization said mixture of ethanol and water 25 from said fermented fine-divided waste 32 and to form ethanol-free fermented fine-divided waste 50 in said separate vaporization vessel 49. The twelfth preferred embodiment of the method of the invention comprises a step for feeding ethanol-free fermented fine-divided waste 50 from the separate vaporization vessel 48 to the separate dehydration vessel 49. This twelfth preferred embodiment of the method of the invention includes a dehydration step performed in the separate dehydration vessel 49 dehydrating said ethanol-free fermented fine-divided waste 50 to form dry matter 24 of said ethanol-free fermented fine-divided waste 50. This twelfth preferred embodiment of the invention includes an ethanol mixture collecting step for discharging and collecting said vaporized mixture of ethanol and water 25 from said separate vaporization vessel 48. This twelfth preferred embodiment of the invention includes a dry matter 24 collecting step for discharging and collecting said dry matter 24 from said separate dehydration vessel 49.

In the same manner as the first preferred embodiment of the method of the invention the twelfth preferred embodiment of the method of the invention may comprises a pre-evaporation step for removing fermentation inhibitors from fermented fine-divided waste containing fermentation inhibitors 47. In the twelfth preferred embodiment of the method of the invention fermentation inhibitors are removed by feeding fermented fine-divided waste containing fermentation inhibitors 47 into the separate vaporization vessel 48 and by using the separate vaporization vessel 48 for vaporizing fermentation inhibitors off the fermented fine-divided waste containing fermentation inhibitors 47.

The method may as in the sixteenth embodiment shown in FIG. 16 that otherwise essentially corresponds to the fifth embodiment shown in FIG. 5, comprise a solid matter separation step that is performed in solid matter separator means 53 after the saccharification step and fermentation step that is performed in the combined hydrolyzing and fermentation vessel 13 and before the vaporization step that is performed in the separate vaporization and dehydration vessel 11. In this fourteenth embodiment of the method of the invention fermented fine divided waste 32 is in the solid matter separator means 53 separated into fermentation beer 55 or ethanol beer that is fed into a evaporator means 54 and into partly dehydrated fermented fine-divided waste 46 that is fed into at least one of the separate vaporization and dehydration vessel 11 to be used in the vaporization step and the dehydrations step and the combined hydrolyzing and fermentation vessel 13 to be used in the saccaharification step and fermentation step. In the evaporator means 54 the fermentation beer 55 or ethanol beer is subjected to a evaporation step for dividing fermentation beer 55 into mixture of ethanol and water 25 that in FIG. 16 is fed to concentration means 26 for performing a concentration step and into a residual bottom stream 56 that is fed into at least one of the separate hydrolyzing vessel 8 to be used in the saccarification step and the combined hydrolyzing and fermentation vessel 13 to be used in the saccharification step and/or the fermentation step.

In a seventh preferred embodiment of the method of the invention shown in FIG. 7 the method includes a feeding step for feeding municipal biodegradable waste 1 into a separate crusher 15.

This seventh preferred embodiment of the method of the invention includes a crushing step including crushing municipal biodegradable waste 1 with said separate crusher 15 to form fine-divided waste 30.

This seventh preferred embodiment of the invention includes a feeding step for feeding said fine-divided waste 30 from said separate crusher 15 into a separate hydrolyzing vessel 8.

This seventh preferred embodiment of the method of the invention includes a saccharification step performed in said separate hydrolyzing vessel 8 for at least partly saccharifying with saccharifying enzymes said fine-divided waste 30 to form saccharified fine-divided waste 31.

This seventh preferred embodiment of the invention includes a feeding step for feeding said saccharified fine-divided waste 31 from said separate hydrolyzing vessel 8 into a combined fermentation and vaporization and dehydration vessel 12.

This seventh preferred embodiment of the method of the invention includes firstly a fermentation step performed in said combined fermentation and vaporization and dehydration vessel 12 for at least partly fermenting with an ethanol-producing microorganism said saccharified fine-divided waste 31 to obtain fermented fine-divided waste 32 containing a mixture of ethanol and water 25 in said combined fermentation and vaporization and dehydration vessel 12.

This seventh preferred embodiment of the invention includes thereafter a vaporization and dehydrating step performed in said combined fermentation and vaporization and dehydration vessel 12 for at least partly separating by vaporization of said mixture of ethanol and water 25 from said fermented fine-divided waste 32 and for dehydrating said fermented fine-divided waste 32 to form dry matter 24 of said fermented fine-divided waste 32.

This seventh preferred embodiment of the method of the invention includes an ethanol mixture collecting step for discharging and collecting said vaporized mixture of ethanol and water 25 from said combined fermentation and vaporization and dehydration vessel 12.

This seventh preferred embodiment of the method of the invention includes a dry matter 24 collecting step for discharging and collecting said dry matter 24 from said combined fermentation and vaporization and dehydration vessel 12.

The fermentation step of this seventh preferred embodiment of the method of the invention illustrated in FIG. 7 may include performing a pre-vaporization step for removing fermentation inhibitors from fermented fine-divided waste containing fermentation inhibitors 47. This pre-vaporization step includes a pre-evaporation step for separating in said combined fermentation and vaporization and dehydration vessel 12 by vaporization fermentation inhibitors from said fine-divided waste containing fermentation inhibitors 47 whereby partly dehydrated fermented fine-divided waste 46 is obtained. The pre-vaporization step may include performing a feeding step for feeding partly dehydrated fermented fine-divided waste 46 from said combined fermentation and vaporization and dehydration vessel 12 into at least one of said separate hydrolyzing vessel 8 and said separate crusher 15. This pre-vaporization step includes preferably a feeding step for feeding partly dehydrated fermented fine-divided waste 46 from said combined fermentation and vaporization and dehydration vessel 12 only to said separate hydrolyzing vessel 8. The pre-vaporization step may include continuing said fermentation step in said combined fermentation and vaporization and dehydration vessel 12 after performing said pre-vaporization step by at least partly fermenting with ethanol-producing microorganism 23 said partly dehydrated fermented fine-divided waste 46 in said combined fermentation and vaporization and dehydration vessel 12.

In an eighth preferred embodiment of the method of the invention shown in FIG. 8 the method includes a feeding step for feeding municipal biodegradable waste 1 into a separate crusher 15.

This eighth preferred embodiment of the method of the invention includes a crushing step including crushing municipal biodegradable waste 1 with said separate crusher 15 to form fine-divided waste 30.

This eighth preferred embodiment of the invention includes a feeding step for feeding said fine-divided waste 30 from said separate crusher 15 into a combined hydrolyzing and fermentation and vaporization and dehydration vessel 14.

This eighth preferred embodiment of the method of the invention includes firstly a saccharification step performed in said combined hydrolyzing and fermentation and vaporization and dehydration vessel 14 for saccharifying with saccharifying enzymes said fine-divided waste 30 to form saccharified fine-divided waste 31.

This eighth preferred embodiment of the method of the invention includes thereafter a fermentation step performed in said combined hydrolyzing and fermentation and vaporization and dehydration vessel 14 for at least partly fermenting with an ethanol-producing microorganism said saccharified fine-divided waste 31 to obtain fermented fine-divided waste 32 containing mixture of ethanol and water 25 in the combined hydrolyzing and fermentation and vaporization and dehydration vessel 14.

This eighth preferred embodiment of the method of the invention includes thereafter a vaporization and dehydrating step performed in said combined hydrolyzing and fermentation and vaporization and dehydration vessel 14 for at least partly separating by vaporization said mixture of ethanol and water 25 from said fermented fine-divided waste 32 and for dehydrating said fermented fine-divided waste 32 to form dry matter 24 of said fermented fine-divided waste 32 in said combined hydrolyzing and fermentation and vaporization and dehydration vessel 14.

This eighth preferred embodiment of the method of the invention includes an ethanol mixture collecting step for discharging and collecting said vaporized mixture of ethanol and water 25 from said combined hydrolyzing and fermentation and vaporization and dehydration vessel 14.

This eighth preferred embodiment of the method of the invention includes a dry matter 24 collecting step for discharging and collecting said dry matter 24 from said combined hydrolyzing and fermentation and vaporization and dehydration vessel 14.

The fermentation step of this eight preferred embodiment of the method of the invention may include a pre-vaporization step for removing fermentation inhibitors from fermented fine-divided waste containing fermentation inhibitors 47. This pre-vaporization step includes pre-vaporization step includes a pre-evaporation step for separating in said combined hydrolyzing and fermentation and vaporization and dehydration vessel 14 by vaporization fermentation inhibitors from said fine-divided waste containing fermentation inhibitors 47 whereby partly dehydrated fermented fine-divided waste 46 is obtained. This pre-vaporization step includes by continuing said fermentation step in said combined hydrolyzing and fermentation and vaporization and dehydration vessel 14 after said pre-vaporization step by at least partly fermenting with ethanol-producing microorganism 23 said partly dehydrated fermented fine-divided waste 46 in said combined hydrolyzing and fermentation and vaporization and dehydration vessel 14.

In a preferred embodiment of the invention said crushing step includes fine dividing of said municipal biodegradable waste 1 into particles having a size of about 0.5 to about 2 mm, preferably about 1 to about 1.5 mm

In a preferred embodiment of the invention said hydrolyzing step including a feeding step for adding acid 21 to said fine-divides waste 30, the acid 21 being selected from organic acid and mineral acid, such as sulfuric acid.

In a preferred embodiment of the invention said hydrolyzing step including a feeding step for adding saccharifying enzymes 20 to said fine-divided waste 30, the saccharifying enzymes 20 being selected from amylases, cellulases, and hemicellulases. Amylases are hydrolytic enzymes, which hydrolyze starch and include α-amylases, β-amylases and glycoamylases. Cellulases hydrolyzing cellulose include endoglucanases, cellobiohydrolases and β-glucosidases. Enzymes degrading hemicellulose include xylanases and mannanases and side-chain cleaving enzymes, such as α-glucuronidases, acetyl xylan esterases, α-arabinofuranosidases and α-galactosidases. After combined action of these enzymes, the starch, cellulose and hemicellulose polymers are hydrolyzed into monomeric hexose and pentose sugars, which then may be fermented by ethanol-producing microorganisms. Different enzyme mixtures for hydrolyzing starch, cellulose and hemicellulose polymers are commercially available.

In a preferred embodiment of the invention said hydrolyzing step including a feeding step for adding other hydrolyzing enzymes 45 to said fine-divided waste 30, the other hydrolyzing enzymes 45 being selected from proteases and pectinases.

In a preferred embodiment of the invention said hydrolyzing step including a feeding step for adding enzymes having both a saccharifying and a hydrolyzing effect to said fine-divided waste 30.

In a preferred embodiment of the invention said hydrolyzing step includes a feeding step for adding water 22 to the biodegradable waste comprising at least one of starch and cellulose.

In a preferred embodiment of the invention said fermentation step includes a feeding step for adding an ethanol-producing microorganism 23 to said saccharified fine-divided waste 31.

In another preferred embodiment of the invention the method includes a burning step for burning said dry matter 24 collected in said dry matter 24 collection step.

In another preferred embodiment of the invention the method includes a concentration step for concentrating said mixture of ethanol and water 25 collected in said ethanol collection step by means of a concentration means 26 for concentrating said mixture of ethanol and water 25.

### EXAMPLE

A pilot scale test was performed with a Drais TurbuDry 250 vacuum dryer (Drais-Werke GmbH, Mannheim, Germany), in other words with a test equipment corresponding to the first preferred embodiment shown in FIG. 1 and described in this application.

250 l municipal biodegradable waste was collected for the test. Polymers and bones were removed manually from the municipal biodegradable waste and the weight of the purified municipal biodegradable waste was after this purification 120 kg. The dry matter content of the purified municipal biodegradable waste was measured to 32%.

The purified municipal biodegradable waste (120 kg) was fed into the vacuum dryer and 60 kg water was added to the purified municipal biodegradable waste, which resulted in a dry matter content of 20%.

Thereafter the mixture of purified municipal biodegradable waste and water was fine-divided by means of a crusher arranged in the vacuum dryer.

Liquefaction enzyme (Genencor: Spezyme Fred, dosage 5×0.14% dry matter) was added to the cold mixture and the temperature of the mixture was raised to 75° C. The separator arranged in the vacuum dryer was turned on during the liquefaction and heating to rotate at a speed of 85 rpm.

After 60 minutes the temperature of the mixture was lowered to a fermentation temperature of 32° C. The revolution of the crusher was lowered to 30 rpm during the fermentation. Yeast (Yeast cream, manufacturer Suomen Hiiva, Finland 8 g/l) and saccharifying enzyme (Genencor, USA: Stargen, dosage 0.28% of the dry matter) was added to the cooled mixture. The interior of the vacuum dryer was nitrified to faster achieve an anaerobic state. The temperature was kept at 32° C. during the fermentation, which was let to continue for 44 hours, after which the fermentation was let to continue, but the fermentation was slower.

After this the fermented mixture was dehydrated by carefully lowering the pressure. The vapor was collected in the beginning until the temperature and the pressure were stabilized. The collected vapor was filtrated and the result was 15 kg of 4.2% ethanol. Dehydration of the mixture was continued without collection of vapor until the dry matter percentage of the mixture was 94% and the result was 14 kg dry matter i.e. solid biofuel. The effective caloric value of the solid biofuel was measured to be 18.48 MJ/kg (5.133 MWh/t).

It is apparent to a person skilled in the art that as technology advances, the basic idea of the invention can be implemented in various ways. It is obvious for a person skilled in the art that the number of vessels can be different from the number of vessels shown in the apparatuses of the examples. It is obvious for a person skilled in the art that the apparatuses can comprise several vessels of the same type for example arranged in series. The invention and its embodiments are therefore not restricted to the above examples, but they may vary within the scope of the claims.

### LIST OF REFERENCE NUMERALS USED

1. Waste
2. Crushing means
3. Hydrolyzing means
4. Fermentation means
5. Vaporization and dehydrating means
6. First discharging means
7. Second discharging means
8. Separate hydrolyzing vessel
9. Crusher
10. Separate fermentation vessel
11. Separate vaporization and dehydration vessel
12. Combined fermentation and vaporization and dehydration vessel
13. Combined hydrolyzing and fermentation vessel
14. Combined hydrolyzing and fermentation and vaporization and dehydration vessel
15. Separate crusher
16. Means for adding saccharifying enzymes
17. Means for adding acid
18. Means for adding water
19. Means for adding ethanol-producing microorganism
20. Saccharifying enzymes
21. Acid
22. Water
23. Ethanol-producing microorganism
24. Dry matter
25. Mixture of ethanol and water
26. Concentration means
27. Distiller colonna
28. Boiler for colonna
29. Distilled water
30. Fine-divided waste
31. Saccharified fine-divided waste
32. Fermented fine-divided waste
33. Hot water or steam out
34. Hot water or steam in
35. Removal of distilled water from the process
36. Heated evaporate
37. Condensed evaporate
38. First heater
39. Concentrated mixture of ethanol and water
40. Burning means
41. Second heater
42. Third heater
43. Mixing means
44. Means for adding hydrolyzing enzymes
45. Hydrolyzing enzymes
46. Partly dehydrated fermented fine-divided waste
47. Fine-divided waste containing fermentation inhibitors
48. Separate vaporization vessel
49. Separate dehydration vessel
50. Ethanol-free fermented fine-divided waste
51. Separate vaporization means
52. Separate dehydration means
53. Solid-matter separator means
54. Evaporator means
55. Fermentation beer
56. Residual bottom stream

1. A method for processing waste (1) containing fermentable raw materials selected from sugars and raw materials such as starch and cellulose capable of being saccharified into fermentable sugars,
characterized by
a crushing step for fine-dividing the waste (1) to form fine-divided waste (30),
a saccharification step for at least partly saccharifying with saccharifying enzymes (20) the fine-divided waste (30) to form fermentable sugars of fermentable raw materials such as starch and cellulose present in the fine-divided waste (30) to obtain saccharified fine-divided waste (31),
a fermentation step for at least partly fermenting with an ethanol-producing microorganism (23) said saccharified fine-divided waste (31) to obtain fermented fine-divided waste (32) containing a mixture of ethanol and water (25),
a vaporization step for at least partly separating by vaporization said mixture of ethanol and water (25) from said fermented fine-divided waste (32),
a dehydrating step for at least partly dehydrating said fermented fine-divided waste (32) to form dry matter (24) of said fermented fine-divided waste (32),
an ethanol mixture collecting step for discharging and collecting said vaporized mixture of ethanol and water (25), and
a dry matter (24) collecting step for discharging and collecting said dry matter (24).

2. The method according to claim 1, characterized
by said fermentation step including a pre-vaporization step for removing fermentation inhibitors from fermented fine-divided waste containing fermentation inhibitors (47) by vaporization fermentation inhibitors from said fermented fine-divided waste containing fermentation inhibitors (47).

3. The method according to claim 1, characterized
by including a feeding step for feeding said waste (1) into a separate hydrolyzing vessel (8),
by said crushing step including crushing said waste (1) with a crusher (9), which is arranged in said separate hydrolyzing vessel (8), and
by performing said saccharification step in said separate hydrolyzing vessel (8).

4. The method according to claim 3, characterized
by performing said fermentation step in a separate fermentation vessel (10),
by performing said dehydration step in a separate vaporization and dehydration vessel (11),
by including a feeding step for feeding saccharified fine-divided waste (31) from said separate hydrolyzing vessel (8) in said separate fermentation vessel (10), and
by including a feeding step for feeding fermented fine-divided waste (32) containing a mixture of ethanol and water (25) from said separate fermentation vessel (10) into said separate vaporization and dehydration vessel (11).

5. The method according to claim 4, characterized
by said fermentation step including a pre-vaporization step for removing fermentation inhibitors from fermented fine-divided waste containing fermentation inhibitors (47), which pre-vaporization step includes a feeding step for feeding fermented fine-divided waste containing fermentation inhibitors (47) from said separate fermentation vessel (10) to the separate vaporization and dehydration vessel (11), a pre-evaporation step for separating by vaporization in the separate vaporization and dehydration vessel (11) fermentation inhibitors from said fine-divided waste containing fermentation inhibitors (47) whereby partly dehydrated fermented fine-divided waste (46) is obtained, and a feeding step for feeding partly dehydrated fermented fine-divided waste (46) from said separate vaporization and dehydration vessel (11) into at least one of the separate hydrolyzing vessel (8) and the separate fermentation vessel (10).

6. The method according to claim 23 characterized
by performing said fermentation step and said dehydration step in a combined fermentation and vaporization and dehydration vessel (12), and
by including a feeding step for feeding saccharified fine-divided waste (31) from said separate hydrolyzing vessel (8) into said combined fermentation and vaporization and dehydration vessel (12).

7. The method according to claim 6, characterized
by said fermentation step including a pre-vaporization step for removing fermentation inhibitors from fermented fine-divided waste containing fermentation inhibitors (47), which pre-vaporization step includes a pre-evaporation step for separating in said combined fermentation and vaporization and dehydration vessel (12) by vaporization fermentation inhibitors from said fine-divided waste containing fermentation inhibitors (47) whereby partly dehydrated fermented fine-divided waste (46) is obtained.

8. The method according to claim 7, characterized
by performing a feeding step for feeding partly dehydrated fermented fine-divided waste (46) from said combined fermentation and vaporization and dehydration vessel (12) into said separate hydrolyzing vessel (8).

9. The method according to claim 7 or 8, characterized
by continuing said fermentation step in said combined fermentation and vaporization and dehydration vessel (12) after performing said pre-vaporization step by at least partly fermenting with ethanol-producing microorganism (23) said partly dehydrated fermented fine-divided waste (46) in said combined fermentation and vaporization and dehydration vessel (12).

10. The method according to claim 1, characterized
by including a feeding step for feeding said waste (1) into a combined hydrolyzing and fermentation vessel (13) for both saccharifying with saccharifying enzymes said fine-divided waste (30) to form saccharified fine-divided waste (31) and fermenting with an ethanol-producing microorganism (23) said saccharified fine-divided waste (31) to form fermented fine-divided waste (32) containing mixture of ethanol and water (25),
by said crushing step including crushing said waste (1) with a crusher (9), which is arranged in said combined hydrolyzing and fermentation vessel (13),
by performing said saccharification step in said combined hydrolyzing and fermentation vessel (13), and
by performing said fermentation step in said combined hydrolyzing and fermentation vessel (13).

11. The method according to claim 10, characterized
by performing said dehydration step in a separate vaporization and dehydration vessel (11), and
by performing a feeding step for feeding fermented fine-divided waste (32) containing mixture of ethanol and water (25) from said combined hydrolyzing and fermentation vessel (13) into said separate vaporization and dehydration vessel (11).

12. The method according to claim 11, characterized
by said fermentation step including a pre-vaporization step for removing fermentation inhibitors from fermented fine-divided waste containing fermentation inhibitors (47), which pre-vaporization step includes a feeding step for feeding fermented fine-divided waste containing fermentation inhibitors (47) from said combined hydrolyzing and fermentation vessel (13) to the separate vaporization and dehydration vessel (11), a pre-evaporation step for separating by vaporization in the separate vaporization and dehydration vessel (11) fermentation inhibitors from fine-divided waste containing fermentation inhibitors (47) whereby partly dehydrated fermented fine-divided waste (46) is obtained, and a feeding step for feeding partly dehydrated fermented fine-divided waste (46) from said separate vaporization and dehydration vessel (11) into said combined hydrolyzing and fermentation vessel (13).

13. The method according to claim 1, characterized
by including a feeding step for feeding said waste (1) into a combined hydrolyzing and fermentation and vaporization and dehydration vessel (14) for saccharifying with saccharifying enzymes said fine-divided waste (30) to form saccharified fine-divided waste (31) and for fermenting with an ethanol-producing microorganism (23) said saccharified fine-divided waste (31) to form fermented fine-divided waste (32) containing mixture of ethanol and water (25) and for at least partly separating by vaporization said mixture of ethanol and water (25) from said fermented fine-divided waste (32) and for dehydrating fermented fine-divided waste (32) to form dry matter (24) of said fermented fine-divided waste (32),
by said crushing step including crushing said waste (1) with a crusher (9), which is arranged in said combined hydrolyzing and fermentation and vaporization and dehydration vessel (14),
by performing said saccharification step in said combined hydrolyzing and fermentation and vaporization and dehydration vessel (14),
by performing said fermentation step in said hydrolyzing and fermentation and vaporization and dehydration vessel (14), and
by performing said vaporization step and said dehydration step in said combined hydrolyzing and fermentation and vaporization and dehydration vessel (14).

14. The method according to claim 13, characterized
by said fermentation step including a pre-vaporization step for removing fermentation inhibitors from fermented fine-divided waste containing fermentation inhibitors (47), which pre-vaporization step includes a pre-evaporation step for separating in said combined hydrolyzing and fermentation and vaporization and dehydration vessel (14) by vaporization fermentation inhibitors from said fine-divided waste containing fermentation inhibitors (47) whereby partly dehydrated fermented fine-divided waste (46) is obtained, and
by continuing said fermentation step in said combined hydrolyzing and fermentation and vaporization and dehydration vessel (14) after said pre-vaporization step by at least partly fermenting with ethanol-producing microorganism (23) said partly dehydrated fermented fine-divided waste (46) in said combined hydrolyzing and fermentation and vaporization and dehydration vessel (14).

15. The method according to claim 1, characterized
by including a feeding step for feeding said waste (1) into a separate crusher (15), and
by said crushing step including crushing said waste (1) with said separate crusher (15) to form fine-divided waste (30).

16. The method according to claim 15, characterized
by including a feeding step for feeding said fine-divided waste (30) from said separate crusher (15) into a combined hydrolyzing and fermentation vessel (13) for both saccharifying with saccharifying enzymes said fine-divided waste (30) to form saccharified fine-divided waste (31) and for fermenting with an ethanol-producing microorganism (23) said saccharified fine-divided waste (31) to form fermented fine-divided waste (32) containing mixture of ethanol and water (25),
by including a feeding step for feeding said fermented fine-divided waste (32) containing a mixture of ethanol and water (25) from said combined hydrolyzing and fermentation vessel (13) into a separate vaporization and dehydration vessel (11) for at least partly separating by vaporization mixture of ethanol and water (25) from said fermented fine-divided waste (32) and for dehydrating said fermented fine-divided waste (32) to form dry matter (24) of said fermented fine-divided waste (32),
by performing both said saccharification step in said combined hydrolyzing and fermentation vessel (13), and
by performing said vaporization step and said dehydration step in said separate vaporization and dehydration vessel (11).

17. The method according to claim 16, characterized
by said fermentation step including a pre-vaporization step for removing fermentation inhibitors from fermented fine-divided waste containing fermentation inhibitor (47), which pre-vaporization step includes a feeding step for feeding fermented fine-divided waste containing fermentation inhibitors (47) from said combined hydrolyzing and fermentation vessel (13) to the separate vaporization and dehydration vessel (11), a pre-evaporation step for separating by vaporization in the separate vaporization and dehydration vessel (11) fermentation inhibitors from said fine-divided waste containing fermentation inhibitors (47) whereby partly dehydrated fermented fine-divided waste (46) is obtained, and a feeding step for feeding partly dehydrated fermented fine-divided waste (46) from said separate vaporization and dehydration vessel (11) into said combined hydrolyzing and fermentation vessel (13) into at least one of said combined hydrolyzing and fermentation vessel (13) and said separate crusher (15).

18. The method according to claim 15, characterized
by including a feeding step for feeding said fine-divided waste (30) from said separate crusher (15) into a separate hydrolyzing vessel (8) for saccharifying with saccharifying enzymes (20) said fine-divided waste (30) to form saccharified fine-divided waste (31),
by including a feeding step for feeding said saccharified fine-divided waste (31) from said separate hydrolyzing vessel (8) into a combined fermentation and vaporization and dehydration vessel (12) for fermenting with an ethanol-producing microorganism (23) said saccharified fine-divided waste (31) to form fermented fine-divided waste (32) containing a mixture of ethanol and water (25) and for at least partly separating by vaporization said mixture of ethanol and water (25) from said fermented fine-divided waste (32) and for dehydrating said fermented fine-divided waste (32) to form dry matter (24) of said fermented fine-divided waste (32),
by performing said saccharification step in said separate hydrolyzing vessel (8), and
by performing said fermentation step, said vaporization step, and said dehydration step in said combined fermentation and vaporization and dehydration vessel (12).

19. The method according to claim 18, characterized
by said fermentation step including a pre-vaporization step for removing fermentation inhibitors from fermented fine-divided waste containing fermentation inhibitors (47), which pre-vaporization step includes a pre-evaporation step for separating in said combined fermentation and vaporization and dehydration vessel (12) by vaporization fermentation inhibitors from said fine-divided waste containing fermentation inhibitors (47) whereby partly dehydrated fermented fine-divided waste (46) is obtained.

20. The method according to claim 19, characterized
by performing a feeding step for feeding partly dehydrated fermented fine-divided waste (46) from said combined fermentation and vaporization and dehydration vessel (12) into at least one of said separate hydrolyzing vessel (8) and said separate crusher (15).

21. The method according to claim 19 or 20, characterized
by continuing said fermentation step in said combined fermentation and vaporization and dehydration vessel (12) after said pre-vaporization step by at least partly fermenting with ethanol-producing microorganism (23) said partly dehydrated fermented fine-divided waste (46) in said combined fermentation and vaporization and dehydration vessel (12).

22. The method according claim 15, characterized
by including a feeding step for feeding said fine-divided waste (30) from said separate crusher (15) into a combined hydrolyzing and fermentation and vaporization and dehydration vessel (14) for saccharifying with saccharifying enzymes (20) said fine-divided waste (30) to form saccharified fine-divided waste (31) and for fermenting with an ethanol-producing microorganism (23) said saccharified fine-divided waste (31) to form fermented fine-divided waste (32) containing mixture of ethanol and water (25) and for at least partly separating by vaporization said mixture of ethanol and water (25) from fermented fine-divided waste (32) and dehydrating said fermented fine-divided waste (32) to form dry matter (24) of said fermented fine-divided waste (32), and
by performing said saccharification step, said fermentation step, said vaporization step, and said dehydration step in said combined hydrolyzing and fermentation and vaporization and dehydration vessel (14).

23. The method according to claim 22, characterized
by said fermentation step including a pre-vaporization step for removing fermentation inhibitors from fermented fine-divided waste containing fermentation inhibitors (47), which pre-vaporization step includes a pre-evaporation step for separating in said combined hydrolyzing and fermentation and vaporization and dehydration vessel (14) by vaporization fermentation inhibitors from said fine-divided waste containing fermentation inhibitors (47) whereby partly dehydrated fermented fine-divided waste (46) is obtained.

24. The method according to claim 23, characterized
by performing a feeding step for feeding partly dehydrated fermented fine-divided waste (46) from said combined hydrolyzing and fermentation and vaporization and dehydration vessel (14) into said separate crusher (15).

25. The method according to claim 23 or 24, characterized
by continuing said fermentation step in said combined hydrolyzing and fermentation and vaporization and dehydration vessel (14) after said pre-vaporization step by at least partly fermenting with ethanol-producing microorganism (23) said partly dehydrated fermented fine-divided waste (46) in said combined hydrolyzing and fermentation and vaporization and dehydration vessel (14).

26. The method according to claim 15, characterized
by including a feeding step for feeding said fine-divided waste (30) from said separate crusher (15) into a separate hydrolyzing vessel (8) for saccharifying with saccharifying enzymes (20) said fine-divided waste (30) to form saccharified fine-divided waste (31),
by including a feeding step for feeding said saccharified fine-divided waste (31) from said separate hydrolyzing vessel (8) into a separate fermentation vessel (10) for fermenting with an ethanol-producing microorganism (23) said saccharified fine-divided waste (31) to form fermented fine-divided waste (32) containing a mixture of ethanol and water (25), and
by including a feeding step for feeding said fermented fine-divided waste (32) containing a mixture of ethanol and water (25) from said separate fermentation vessel (10) into a separate vaporization and dehydration vessel (11) for at least partly separating by vaporization said mixture of ethanol and water (25) from said fermented fine-divided waste (32) and for dehydrating said fermented fine-divided waste (32) to form dry matter (24) of said fermented fine-divided waste (32)
by performing said saccharification step in said separate hydrolyzing vessel (8),
by performing said fermentation step in said separate fermentation vessel (10), and
by performing said vaporization step and said dehydration step in said separate vaporization and dehydration vessel (11).

27. The method according to claim 26, characterized
by said fermentation step including a pre-vaporization step for removing fermentation inhibitors from fermented fine-divided waste containing fermentation inhibitors (47), which pre-vaporization step includes a feeding step for feeding fermented fine-divided waste containing fermentation inhibitors (47) from said separate fermentation vessel (10) to the separate vaporization and dehydration vessel (11), a pre-evaporation step for separating by vaporization in the separate vaporization and dehydration vessel (11) fermentation inhibitors from said fine-divided waste containing fermentation inhibitors (47) whereby partly dehydrated fermented fine-divided waste (46) is obtained, and a feeding step for feeding partly dehydrated fermented fine-divided waste (46) from said separate vaporization and dehydration vessel (11) into at least one of said separate fermentation vessel (10), said separate hydrolyzing vessel (8) and said separate crusher (15).

28. The method according to any of the claims 1 to 27, characterized by said crushing step including fine dividing of said waste (1) into particles having a size of about 0.5 to about 2 mm, preferably about 1 to about 1.5 mm.

29. The method according to any of the claims 1 to 28, characterized by said saccharification step including a feeding step for adding saccharifying enzymes (20) to said fine-divided waste (30), the saccharifying enzymes (20) being selected from amylases, cellulases and hemicellulases.

30. The method according to any of the claims 1 to 29, characterized by said saccharification step including a feeding step for adding hydrolyzing enzymes (45) to said fine-divided waste (30), the hydrolyzing enzymes (45) being selected from proteases and pectinases.

31. The method according to any of the claims 1 to 30, characterized by said saccharification step including a feeding step for adding liquid such as water to the biodegradable waste comprising at least one of starch and cellulose.

32. The method according to any of the claims 1 to 31, characterized by said fermentation step including a feeding step for adding an ethanol-producing microorganism (23), preferably Saccharomyces ceravisiae, to said saccharified fine-divided waste (31).

33. The method according to any of the claims 1 to 32, characterized
by including a burning step for burning said dry matter (24) collected in said dry matter (24) collection step to produce thermal energy, and
by using said thermal energy used in at least one of said saccharification step, said fermentation step, said vaporization step and said dehydration step.

34. The method according to any of the claims 1 to 33, characterized by including a concentration step for concentrating said mixture of ethanol and water (25) collected in said ethanol collection step by means of a concentration means (26) for concentrating said mixture of ethanol and water (25) to form concentrated mixture of ethanol and water (39).

35. The method according to any of the claims 1 to 34, characterized by mixing and/or moving said fermented fine-divided waste (32) during at least one of the vaporization step and the dehydrating step.

36. The method according to any of the claims 1 to 35, characterized
by a solid matter separation step that is performed after the fermentation step and before the vaporization step, in which solid matter separation step fermentation beer (55) is separated from fermented fine-divided waste (32), and
by a evaporation step for separating mixture of ethanol and water (25) by evaporation from fermentation beer (55) obtained in the solid matter separation step.

37. The method according to claim 36, characterized by using partly dehydrated fermented fine-divided waste (46) obtained in said solid matter separation step by separating fermentation beer (55) from fermented fine-divided waste (32) in at least one of the following: said crushing step, said saccharification step, said fermentation step, said vaporization step, and said dehydrating step.

38. The method according to claim 36 or 37, characterized by using residual bottom stream (46) obtained in said evaporation step by separating mixture of ethanol and water (25) from fermentation beer (55) in at least one of the following: said crushing step, said saccharification step, and said fermentation step.

39. The method according to any of the claims 36 to 38, characterized by including a concentration step for concentrating said mixture of ethanol and water (25) obtained in said evaporation step by means of a concentration means (26) for concentrating said mixture of ethanol and water (25) to form concentrated mixture of ethanol and water (39).

40. An apparatus for processing waste (1) containing fermentable raw materials selected from sugars and raw materials such as starch and cellulose capable of being saccharified into fermentable sugars, characterized in that said apparatus comprising:
crushing means (2) for at least partly fine-dividing said waste (1) to form fine-divided waste (30),
hydrolyzing means (3) for at least partly saccharifying with saccharifying enzymes (20) said fine-divided waste (30) to form saccharified fine-divided waste (31),
fermentation means (4) for at least partly fermenting with an ethanol-producing microorganism (23) said saccharified fine-divided waste (31) to form fermented fine-divided waste (32) containing a mixture of ethanol and water (25),
vaporization and dehydrating means (5) for at least partly separating by vaporization said mixture of ethanol and water (25) from said fermented fine-divided waste (32) and to form dry matter (24) of said fermented fine-divided waste (32),
first discharging means (6) for discharging said vaporized mixture of ethanol and water (25) from said vaporization and dehydrating means (5), and
second discharging means (7) for discharging said dry matter (24) from said vaporization and dehydrating means (5).

41. The apparatus according to claim 40, characterized
in that said hydrolyzing means (3) comprises a separate hydrolyzing vessel (8), and
in that said crushing means (2) is a crusher (9) arranged in said separate hydrolyzing vessel (8).

42. The apparatus according to claim 41, characterized
in said fermentation means (4) comprises a separate fermentation vessel (10), and
in that said vaporization and dehydrating means (5) comprises a separate vaporization and dehydration vessel (11).

43. The apparatus according to claim 42, characterized
by conduit means for feeding fine-divided waste containing fermentation inhibitors (47) from said separate fermentation vessel (10) to said separate vaporization and dehydration vessel (11).

44. The apparatus according to claim 42 or 43, characterized
by conduit means for feeding partly dehydrated fermented fine-divided waste (46) from said separate vaporization and dehydration vessel (11) to at least one of said separate vaporization and dehydration vessel (11) and said separate fermentation vessel (10).

45. The apparatus according to claim 40, characterized
in that said fermentation means (4) and said vaporization and dehydrating means (5) comprises a combined fermentation and vaporization and dehydration vessel (12).

46. The apparatus according to claim 45, characterized
by conduit means for feeding partly dehydrated fermented fine-divided waste (46) from said combined fermentation and vaporization and dehydration vessel (12) to said separate hydrolyzing vessel (8).

47. The apparatus according to claim 40, characterized
in that said hydrolyzing means (3) and said fermentation means (4) comprise a combined hydrolyzing and fermentation vessel (13), and
in that said crushing means (2) is a crusher (9) arranged in said combined hydrolyzing and fermentation vessel (13).

48. The apparatus according to claim 47, characterized
in that said vaporization and dehydrating means (5) comprises a separate vaporization and dehydration vessel (11).

49. The apparatus according to claim 48, characterized
by conduit means for feeding fine-divided waste containing fermentation inhibitors (47) from said combined hydrolyzing and fermentation vessel (13) to said separate vaporization and dehydration vessel (11).

50. The apparatus according to claim 48 or 49, characterized
by conduit means for feeding partly dehydrated fine-divided waste (46) from said separate vaporization and dehydration vessel (11) to said combined hydrolyzing and fermentation vessel (13).

51. The apparatus according to claim 40, characterized
in that said hydrolyzing means (3), said fermentation means (4), and said vaporization and dehydrating means (5) comprise a combined hydrolyzing and fermentation and vaporization and dehydration vessel (14),
in that said crushing means (2) is a crusher (9) arranged in said combined hydrolyzing and fermentation and vaporization and dehydration vessel (14).

52. The apparatus according to claim 40, characterized
in that said crushing means (2) comprises a separate crusher (15).

53. The apparatus according to claim 52, characterized
in that said hydrolyzing means (3) and said fermentation means (4) comprise a combined hydrolyzing and fermentation vessel (13), and
in that said vaporization and dehydrating means (5) comprises a separate vaporization and dehydration vessel (11).

54. The apparatus according to claim 53, characterized
by conduit means for feeding fine-divided waste containing fermentation inhibitors (47) from said separate fermentation vessel (10) to said separate vaporization and dehydration vessel (11).

55. The apparatus according to claim 53 or 54, characterized
by conduit means for feeding partly dehydrated fermented fine-divided waste (46) from said separate vaporization and dehydration vessel (11) to at least one of said combined hydrolyzing and fermentation vessel (13) and said separate crusher (15).

56. The apparatus according to claim 52, characterized
in that said hydrolyzing means (3) comprises a separate hydrolyzing vessel (8), and
in that said fermentation means (4) and said vaporization and dehydrating means (5) comprise a combined fermentation and vaporization and dehydration vessel (12).

57. The apparatus according to claim 56, characterized
by conduit means for feeding partly dehydrated fermented fine-divided waste (46) from said combined fermentation and vaporization and dehydration vessel (12) to at least one of said separate hydrolyzing vessel (8) and said separate crusher (15).

58. The apparatus according claim 52, characterized
in that said hydrolyzing means (3), said fermentation means (4), and said vaporization and dehydrating means (5) comprise a combined hydrolyzing and fermentation and vaporization and dehydration vessel (14).

59. The apparatus according to claim 52, characterized
in that said hydrolyzing means (3) comprises a separate hydrolyzing vessel (8),
in that said fermentation means (4) comprises a separate fermentation vessel (10), and
in that said vaporization and dehydrating means (5) comprises a separate vaporization and dehydration vessel (11).

60. The apparatus according to claim 59, characterized
by conduit means for feeding partly dehydrated fermented fine-divided waste (46) from said separate vaporization and dehydration vessel (11) to at least one of said separate hydrolyzing vessel (8), said separate fermentation vessel (10), and said separate crusher (15).

61. The apparatus according to any of the claims 40 to 60, characterized
by comprising a concentration means (26) for concentrating said mixture of ethanol and water (25), and
in that said concentration means (26) is in fluid connection with said first discharging means (6) for discharging said vaporized mixture of ethanol and water (25) from said vaporization and dehydrating means (5).

62. The apparatus according to any of the claims 40 to 61, characterized by said vaporization and dehydrating means (5) comprising means for mixing and/or moving said fermented fine-divided waste (32) in said vaporization and dehydrating means (5).

63. The apparatus according to any of the claims 40 to 62, characterized by said vaporization and dehydrating means (5) comprising a rotary dryer.

64. The apparatus according to any of the claims 40 to 63, characterized
by comprising a burning means (40) for receiving dry matter (24) and for producing of thermal energy by burning said dry matter (24),
in that said burning means (40) is functionally connected to said second discharging means (7) for discharging said dry matter (24) from said vaporization and dehydrating means (5), and
in that the apparatus comprises means for feeding at least partly said thermal energy produced by said burning means (40) to at least one of said hydrolyzing means (3), said fermentation means (4), and said vaporization and dehydrating means (5).

65. The apparatus according to any of the claims 40 to 64, characterized
by a solid matter separation means (53) that is arranged for receiving fermented fine-divided waste (32) from the fermentation means (4) and configured for separating fermentation beer (55) from fermented fine-divided waste (32), and
by a evaporator means (54) that is arranged for receiving fermentation beer (55) from the solid matter separation means (53) and configured for separating mixture of ethanol and water (25) from fermentation beer (55).

66. The apparatus according to claim 65, characterized by the apparatus being configured for feeding partly dehydrated fermented fine-divided waste (46) obtained in said solid matter separation means (53) by separating fermentation beer (55) from fermented fine-divided waste (32) into at least one of the following: said crushing means (2), said hydrolyzing means (3), said fermentation means (4), and vaporization and dehydrating means (5).

67. The apparatus according to claim 65 or 66, characterized by the apparatus being configured for feeding residual bottom stream (46) obtained in said evaporator means (54) by separating mixture of ethanol and water (25) from fermentation beer (55) into at least one of the following: said crushing means (2), said hydrolyzing means (3), and said fermentation means (4).

68. The apparatus according to any of the claims 65 to 67, characterized
by comprising a concentration means (26) for concentrating said mixture of ethanol and water (25), and
in that said concentration means (26) is in fluid connection with said evaporator means (54)).

69. Use of the method according to any of the claims 1 to 39 or the apparatus of any of the claims 40 to 68 for the production of ethanol from municipal biodegradable waste.
